(19) United States
(12) Patent Application Publication
(10) Pub. No.: US 2025/0281127 A1
(43) Pub. Date: Sep. 11, 2025

(54) IDENTIFICATION OF DISORDERED BREATHING DURING SLEEP

(71) Applicant: Medtronic, Inc., Minneapolis, MN (US)

(72) Inventors: Juliana E. Pronovici, New Hope, MN (US); Shantanu Sarkar, Roseville, MN (US); Steven G. Nelson, Raynham, MA (US); Bruce D. Gunderson, Plymouth, MN (US); Gautham Rajagopal, Minneapolis, MN (US); Jason C. Lee, Edina, MN (US); Geert Morren, Vissenaken (BE); Trent M. Fischer, St. Paul, MN (US); Yong Cho, Excelsior, MN (US)

(21) Appl. No.: 18/857,276

(22) PCT Filed: Apr. 10, 2023
(86) PCT No.: PCT/IB2023/053637

§ 371 Date: Oct. 16, 2024

Related U.S. Application Data
(60) Provisional application No. 63/363,455, filed on Apr. 22, 2022.

(51) Int. Cl. A61B 5/00; A61B 5/0205; A61B 5/113; A61B 5/318

(52) U.S. Cl. CPC A61B 5/746; A61B 5/0205; A61B 5/113; A61B 5/318; A61B 5/4818; A61B 5/7264; A61B 5/7275; A61B 2562/0219

(57) ABSTRACT

In some examples, a system includes a medical device including one or more sensors configured to sense one or more physiological parameters of a patient. The system also includes processing circuitry configured to: sense, by the one or more sensors, one or more sensor signals indicative of the one or more physiological parameters of the patient; determine, based at least in part on the one or more sensor signals, a waveform corresponding to a breathing pattern of the patient; determine, based on the waveform, an envelope signal; determine a sleep disordered breathing index based at least in part on the envelope signal; and determine a heart condition status of the patient based on the sleep disordered breathing index.

This application claims the benefit of U.S. Provisional Patent Application Ser. No. 63/363,455, filed Apr. 22, 2022, the entire content of which is incorporated herein by reference.

### FIELD

The disclosure relates to medical devices and, more particularly, medical devices for detecting or monitoring heart conditions.

### BACKGROUND

A variety of medical devices have been used or proposed for use to deliver a therapy to and/or monitor a physiological condition of patients. As examples, such medical devices may deliver therapy and/or monitor conditions associated with the heart, muscle, nerve, brain, stomach or other organs or tissue. Medical devices that deliver therapy include medical devices that deliver one or both of electrical stimulation or a therapeutic agent to the patient. Some medical devices have been used or proposed for use to monitor heart failure or to detect heart failure events.

Heart failure is the most common cardiovascular disease that causes significant economic burden, morbidity, and mortality. In the United States alone, roughly 5 million people have heart failure, accounting for a significant number of hospitalizations. Heart failure may result in cardiac chamber dilation, increased pulmonary blood volume, and fluid retention in the lungs. Generally, the first indication that a physician has of heart failure in a patient is not until it becomes a physical manifestation with swelling or breathing difficulties so overwhelming as to be noticed by the patient who then proceeds to be examined by a physician. This is undesirable since hospitalization at such a time would likely be required for a heart failure patient to remove excess fluid and relieve symptoms.

### SUMMARY

This disclosure describes techniques for providing an early warning for various types of sleep disordered breathing (e.g., obstructive sleep apnea, Cheyne-Stokes respiration, central sleep apnea, etc.), as well as various heart conditions (e.g., heart failure decompensation, worsening heart failure, etc.) based on sensed patient physiological parameters corresponding to breathing patterns of the patient. A device, such as a wearable medical device, a subcutaneous implantable medical device (IMD), or a computing device in communication with the IMD, continuously (e.g., on a constant, periodic, or triggered basis without user intervention) monitors a waveform that varies based on respiration. The device may identify respiration in the waveform and identify sleep disordered breathing patterns based on the timing or amplitude of respirations. Measuring occurrences of sleep disordered breathing continuously can prove as a marker for increased risk for impending worsening heart failure or development of arrhythmias such as atrial fibrillation. The device may perform operations based on the identification of sleep disordered breathing in the patient. Example actions include storage, transmission, and/or display of waveform, heart failure risk, or other data for sleep disordered breathing episodes.

The techniques of this disclosure may be implemented by systems including an IMD and that can autonomously and continuously collect physiological parameter data while the IMD is subcutaneously implanted in a patient over months or years and perform numerous operations per second on the data to enable the systems herein to determine varying risk levels of the cardiac event and associated exercise tolerance threshold. Using techniques of this disclosure with an IMD may be advantageous when a physician cannot be continuously present with the patient over weeks or months to evaluate the physiological parameters and/or where performing the operations on the data described herein (signal processing of various respiration signals to identify sleep disordered breathing metrics) could not practically be performed in the mind of a physician. Using the techniques of this disclosure with autonomously/continuously operating IMDs and computing devices may provide a clinical advantage in timely detecting changes in a patient's condition providing timely alerts to the patient and/or caregiver.

In some examples, a system includes a medical device including one or more sensors configured to sense one or more physiological parameters of a patient. The medical system also includes processing circuitry configured to: sense, by the one or more sensors, one or more sensor signals indicative of the one or more physiological parameters of the patient; determine, based at least in part on the one or more sensor signals, a waveform corresponding to a breathing pattern of the patient; determine, based on the waveform, an envelope signal; determine a sleep disordered breathing index based at least in part on the envelope signal; and determine a heart condition status of the patient based on the sleep disordered breathing index.

In some examples, a method includes sensing, by one or more sensors of a medical device, one or more sensor signals indicative of one or more physiological parameters of a patient; determining, by processing circuitry of the medical device, based at least in part on the one or more sensor signals, a waveform corresponding to a breathing pattern of the patient; determining, based on the waveform, an envelope signal; determining a sleep disordered breathing index based at least in part on the envelope signal; and determining a heart condition status of the patient based on the sleep disordered breathing index.

The disclosure also provides means for performing any of the techniques described herein, as well as non-transitory computer-readable media including instructions that cause a programmable processor to perform any of the techniques described herein.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient, in accordance with one or more techniques of this disclosure.

FIG. 2 is a conceptual side-view diagram illustrating an implantable medical device (IMD) of the medical system of FIG. 1 in a subcutaneous space, in accordance with one or more techniques of this disclosure.

FIG. 3 is a functional block diagram illustrating an example configuration of the IMD of FIGS. 1 and 2, in accordance with one or more techniques of this disclosure.

FIG. 4 is a functional block diagram illustrating an example configuration of the external device of FIG. 1, in accordance with one or more techniques of this disclosure.

FIG. 5 is a block diagram illustrating an example configuration of a health monitoring system that operates in accordance with one or more techniques of the present disclosure.

FIG. 6 is a block diagram illustrating an example system that includes an access point, a network, external computing devices, such as a server, and one or more other computing devices, which may be coupled to the IMD and external devices of FIGS. 1-4, in accordance with one or more techniques of this disclosure.

FIG. 7A is a graph illustrating an example subcutaneous tissue impedance signal, in accordance with one or more techniques of this disclosure.

FIG. 7B is a graph illustrating example signals derived from the example subcutaneous tissue impedance signal of FIG. 7A, in accordance with one or more techniques of this disclosure.

FIG. 7C is a graph illustrating an example phase plot derive from the example envelope signal of FIG. 7B, in accordance with one or more techniques of this disclosure.

FIG. 8 is a flow diagram illustrating an example operation for determining a heart condition status of a patient based on a subcutaneous tissue impedance signal, in accordance with one or more techniques of this disclosure.

FIG. 9 is a flow diagram illustrating an example operation for initiating a sleep study mode in an IMD, in accordance with one or more techniques of this disclosure.

FIG. 10A is a perspective drawing illustrating an example IMD.

FIG. 10B is a perspective drawing illustrating another example IMD.

Like reference characters denote like elements throughout the description and figures.

### DETAILED DESCRIPTION

Heart failure (HF) is a chronic, progressive condition in which the heart muscle is unable to pump enough blood to meet the body's needs for blood and oxygen. Compensatory mechanisms activate in response to changes in oxygen demand. One such situation is Cheyne-Stokes Respiration (CSR) in which the patient goes through periods of none, or shallow breathing followed by periods of deeper breaths. A similar mechanism takes place in case of sleep apnea. CSR and other types of sleep disordered breathing (SDB) are observed in patients with severe HF. For example, patients with HF or at risk of HF may experience a number of changes to breathing during sleep, including but not limited to, obstructive sleep apnea, central sleep apnea, Cheyne-Stokes breathing, and paroxysmal nocturnal dyspnea (PND). Sleep apnea and other types of SDB may similarly lead to patient harm or death. These conditions are typically diagnosed through a sleep study.

Measuring occurrences of SDB according to the techniques of this invention can prove as a marker for increased risk for different types of SDB (e.g., sleep apnea) and for impending worsening HF or development of arrhythmias such as atrial fibrillation without the need for an in-person sleep study. A medical device, such as an implantable medical device (IMD) or other device capable of measuring occurrences of SDB may detect episodes of SDB when patient is outside the clinic and engaged in their normal daily life, e.g., continuously, rather than only during a sleep study, and alert the patient or a physician when SDB episodes are detected or the detection of SDB episodes satisfy certain criteria indicating a change in patient health. Earlier detection of SDB episodes may allow physicians to prescribe continuous positive airway pressure (CPAP) or other therapy to patients earlier, reducing instances of heart failure, hospitalization, and death.

According to the techniques of this disclosure, processing circuitry of a system that includes a medical device, e.g., an insertable cardiac monitor, that monitors a comorbid condition such as HF could also detect episodes of SDB conditions by monitoring an impedance waveform or other sensor signal (e.g., an electrocardiogram), that varies based on respiration. The processing circuitry may identify respiration in the impedance or other waveform, identify SDB based on the respiration, e.g., based on identifying predefined patterns in the respiration timing and/or amplitude, and take actions based on the identification of SDB. Example actions include storage, transmission, and/or display of waveform or other data for SDB episodes. In some examples, the processing circuitry may determine a SDB index based on identification of SDB episodes. In some examples, the processing circuitry may determine a risk of worsening HF, e.g., a probability of worsening HF occurring within a predefined future time period, based on the identification of SDB episodes, e.g., the SDB index, in some examples in conjunction with other physiological parameter data of the patient and/or other patient data. In some examples the SDB index may be calibrated to the Apnea Hypopnea Index (AHI).

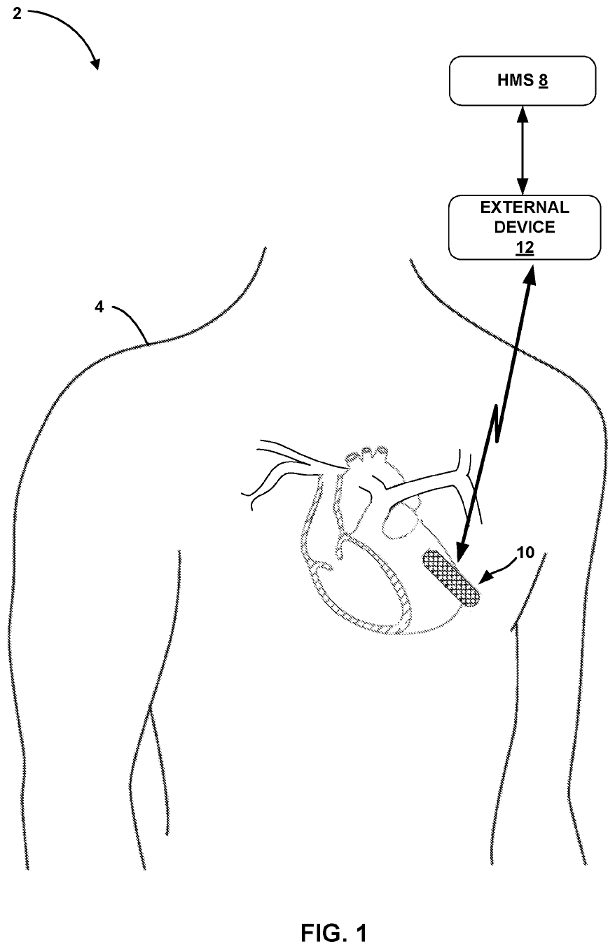

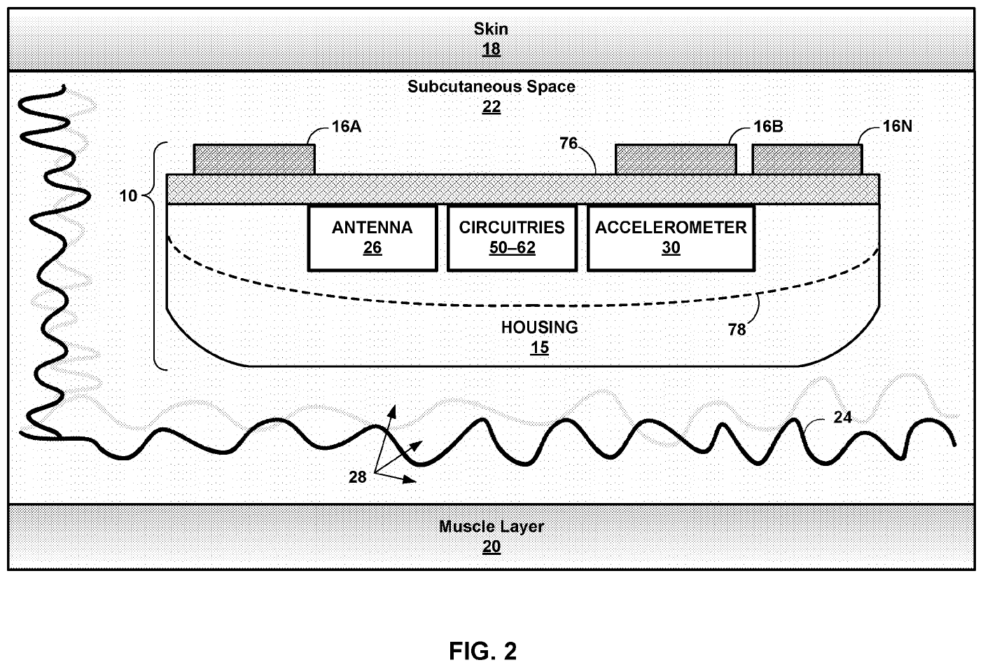

Identification of SDB episodes may include determining an envelope of the respiration cycles in the impedance or other waveform, and identifying patterns, e.g., waxing/waning, within the envelope. In some examples respiration cycles may be measured through impedance measurements. For example, the impedance measured in the subcutaneous tissue, e.g., of the thoracic region, may increase as the patient inhales and will decrease as the patient exhales due to changes in venous return related to the changes of intrathoracic pressure during the respiratory cycle. In general, impedance measurements may be taken via electrodes in the subcutaneous space, e.g., electrodes on a subcutaneously implanted medical device as shown in FIGS. 1 and 2, may be measurements of the impedance of interstitial fluid and subcutaneous tissue. In some examples, respiration cycles may be measured through ECG signals. For example, an R-wave amplitude and/or RR intervals of the ECG signal of a patient may vary with the respiration cycle.

Implantable medical devices (IMDs) may sense and monitor impedance signals and use those signals to determine one or more respiration cycles and/or a heart condition status of a patient or other health condition status of a patient (e.g., edema, sleep apnea, preeclampsia, hypertension, etc.). The electrodes used by IMDs to sense impedance signals are typically integrated with a housing of the IMD and/or coupled to the IMD via one or more elongated leads. Example IMDs that include electrodes include the Reveal LINQ™ or LINQ II™ Insertable Cardiac Monitor (ICM), developed by Medtronic, Inc., of Minneapolis, MN, which may be inserted subcutaneously. Such IMDs may facilitate relatively longer-term monitoring of patients during normal daily activities and may periodically transmit collected data to a network service, such as the Medtronic CareLink® Network, developed by Medtronic, Inc., of Minneapolis, MN.

Medical devices configured to measure impedance via implanted electrodes, including the examples identified herein, may implement the techniques of this disclosure for measuring impedance changes in the interstitial fluid of a patient based on breathing patterns of the patient to determine whether the patient is at risk of different types of SDB (e.g., sleep apnea) as well as HF or arrhythmias such as atrial fibrillation. The techniques include evaluation of the impedance values using criteria configured to provide a desired sensitivity and specificity of SDB detection. The techniques of this disclosure for identifying SDB may facilitate determinations of cardiac wellness and risk of sudden cardiac death and may lead to clinical interventions to suppress sleep apnea or other SDB episodes, or to suppress HF worsening, such as with medications.

FIG. 1 illustrates the environment of an example medical system 2 in conjunction with a patient 4, in accordance with one or more techniques of this disclosure. Patient 4 ordinarily, but not necessarily, will be a human. For example, patient 4 may be an animal needing ongoing monitoring for cardiac conditions. System 2 includes IMD 10. IMD 10 may include one or more sensors configured to sense one or more physiological parameters of patient 4, as well as processing circuitry configured to sense, by the one or more sensors, one or more sensor signals indicative of the one or more physiological parameters of the patient. For example, IMD 10 may include one or more electrodes (not shown) on its housing, or may be coupled to one or more leads that carry one or more electrodes. System 2 may also include external device 12 in communication with IMD 10. Although described mainly with respect to IMD 10, in some examples, some of the methods of the disclosure may be performed by processors of one or more of external device 12 and/or another computing device of system 2 in communication with IMD 10 and/or external device 12. For example, IMD 10 may take one or more measurements and transmit the one or more measurements to external device 12 for analysis by external device or another computing device that communicates with external device 12. Example system 2 may be used to measure subcutaneous impedance to detect episodes of SDB in patient 4 and assess a risk of SDB and/or HF for patient 4.

The example techniques may be used with an IMD 10 of system 2, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 may be implanted anywhere that tissue impedance signals may be measured from the patient. In some examples, IMD 10 may be a wearable medical device, and leads may enter the patient to measure a tissue impedance of the patient. IMD 10 may include a plurality of electrodes (not shown in FIG. 1). Accordingly, IMD 10 may include a plurality of electrodes and may be configured for subcutaneous implantation outside of a thorax of patient 4.

IMD 10 may be configured to measure impedance values within the interstitial fluid of patient 4. For example, IMD 10 may be configured to receive one or more signals indicative of subcutaneous tissue impedance from the electrodes. In some examples, IMD 10 may be a purely diagnostic device. For example, IMD 10 may be a device that only measures subcutaneous impedance values of patient 4. IMD 10 may also use the impedance value measurements to determine one or more impedance waveforms, envelope signals, SDB indexes, heart condition statuses, and/or various thresholds therefor.

Subcutaneous impedance may be measured by delivering a signal through an electrical path between electrodes (not shown in FIG. 1). In some examples, the housing of IMD 10 may be used as an electrode in combination with electrodes located on leads. For example, system 2 may measure subcutaneous impedance by creating an electrical path between a lead and one of the electrodes. In additional examples, system 2 may include an additional lead or lead segment having one or more electrodes positioned subcutaneously or within the subcutaneous layer for measuring subcutaneous impedance. In some examples, two or more electrodes useable for measuring subcutaneous impedance may be formed on or integral with the housing of IMD 10.

In some examples, IMD 10 may also sense an ECG of patient 4 or cardiac electrogram (EGM) signals via the plurality of electrodes and/or operate as a therapy delivery device. For example, IMD 10 may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances. In some examples, system 2 may include any suitable number of leads coupled to IMD 10, and each of the leads may extend to any location within or proximate to a heart or in the chest of patient 4. For example, other examples therapy systems may include three transvenous leads and an additional lead located within or proximate to a left atrium of a heart. As other examples, a therapy system may include a single lead that extends from IMD 10 into a right atrium or right ventricle, or two leads that extend into a respective one of a right ventricle and a right atrium.

In some examples, IMD 10 may be implanted subcutaneously in patient 4. Furthermore, in some examples, external device 12 may monitor subcutaneous impedance values according to the techniques described herein. In some examples, IMD 10 takes the form of the Reveal LINQ™ or LINQ II™ ICM, or another ICM similar to, e.g., a version or modification of, the LINQ™ or LINQ II™ ICM, which may be inserted subcutaneously. Such IMDs may facilitate relatively longer-term monitoring of patients during normal daily activities, and may periodically transmit collected data to a network service, such as the Medtronic CareLink® Network. In the example illustrated by FIG. 1, health monitoring service (HMS) 8 may be a network service that receives data collected by IMD 10.

External device 12 may be a computing device with a display viewable by a user and an interface for providing input to external device 12 (e.g., a user input mechanism). The user may be a physician technician, surgeon, electrophysiologist, clinician, or patient 4. In some examples, external device 12 may be a notebook computer, tablet computer, computer workstation, one or more servers, smartphone or smart watch, personal digital assistant, handheld computing device, smart home device, Internet of Things (IoT) device, networked computing device, or another computing device that may run an application that enables the computing device to interact with IMD 10. External device 12 is configured to communicate with IMD 10 and, optionally, another computing device (not illustrated in FIG. 1), via wired or wireless communication. External device 12, for example, may communicate via near-field communication (NFC) technologies (e.g., inductive coupling, NFC or other communication technologies operable at ranges less than 10-20 cm) and far-field communication technologies (e.g., Radio Frequency (RF) telemetry according to the 802.11 or Bluetooth® specification sets, or other communication technologies operable at ranges greater than NFC technologies). In some examples, external device 12 may include a programming head that may be placed proximate to the body of patient 4 near the IMD 10 implant site in order to improve the quality or security of communication between IMD 10 and external device 12.

External device 12 may be coupled to external electrodes, or to implanted electrodes via percutaneous leads. In some examples, external device 12 may monitor subcutaneous tissue impedance measurements from IMD 10, according to the techniques described herein.

The user interface of external device 12 may receive input from the user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. External device 12 can additionally or alternatively include a peripheral pointing device, such as a mouse, via which the user may interact with the user interface. In some examples, a display of external device 12 may include a touch screen display, and a user may interact with external device 12 via the display. It should be noted that the user may also interact with external device 12 remotely via a networked computing device.

External device 12 may be used to configure operational parameters for IMD 10. For example, external device 12 may provide a parameter resolution for IMD 10 that indicates a resolution of data that IMD 10 should be obtaining. Examples of resolution parameters may include a frequency at which the electrodes process impedance measurements or a frequency at which impedance measurements should be considered in detecting SDB episodes and determining the SDB index and/or HF risk of a patient. In some examples, resolution parameters include filters that specify what type of data or quality of data should flow into these determinations. For example, the type of data may specify that the impedance measurements collected during a certain time period (e.g., daytime, nighttime, high activity, low activity, etc.) should be excluded from the determination, such as by determining statistical representations of historical data through use of non-excluded data. The quality of data may refer to any characteristic used to characterize obtained signal measurements, such as signal-to-noise ratios (SNRs), duplicate data entries, weak signal readings, etc.

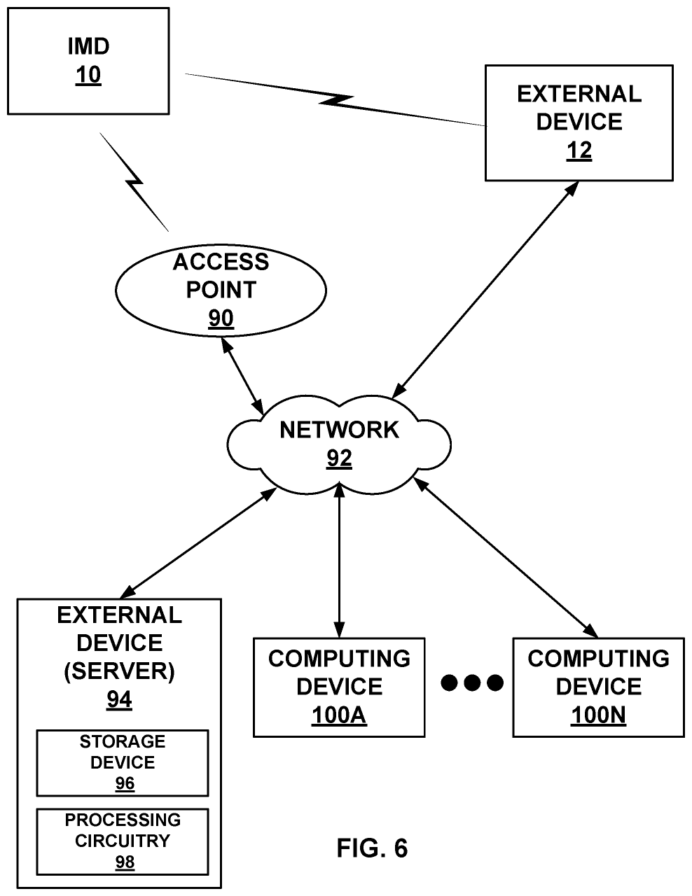

External device 12 may be used to retrieve data from IMD 10. The retrieved data may include impedance values measured by IMD 10, impedance waveforms determined by IMD 10, quantifications of SDB episodes detected by IMD 10, SDB indexes determined by IMD 10, values of other physiological parameters measured by IMD 10, indications of episodes of arrhythmia or other maladies detected by IMD 10, and physiological signals recorded by IMD 10. For example, external device 12 may retrieve information related to detection of one or more SDB episodes, e.g., over a time period since the last retrieval of information by external device 12. External device 12 may also retrieve cardiac EGM segments recorded by IMD 10, e.g., due to IMD 10 determining that an episode of arrhythmia or another malady occurred during the segment, or in response to a request to record the segment from patient 4 or another user. In other examples, the user may also use external device 12 to retrieve information from IMD 10 regarding other sensed physiological parameters of patient 4, such as activity or posture. As discussed in greater detail below with respect to FIG. 6, one or more remote computing devices may interact with IMD 10, e.g., via HMS, in a manner similar to external device 12, e.g., to program IMD 10 and/or retrieve data from IMD 10, via a network.

Processing circuitry of medical system 2, e.g., of IMD 10, external device 12, HMS 8, and/or of one or more other computing devices, may be configured to perform the example techniques of this disclosure for sensing signals (e.g., measuring subcutaneous impedance values, e.g., in the interstitial fluid), to determine waveforms corresponding to breathing patterns of patient 4, to determine envelope signals, to detect SDB episodes, to determine SDB indexes, to determine risk of HF. In some examples, the processing circuitry of medical system 2 analyzes impedance values sensed by IMD 10 to determine whether a waveform is indicative of an SDB episode.

Although described in the context of examples in which IMD 10 includes an insertable or implantable IMD, example systems including one or more external devices of any type configured to sense cutaneous tissue impedances may be configured to implement the techniques of this disclosure. In some examples, radar based systems may sense fluid shifts in patient 4. In some examples, IMD 10 or an external device 12 may use one or more of subcutaneous tissue impedance measurements and intra-vascular impedance. In some examples, processing circuitry of the external device or of IMD 10 may receive intra-vascular impedance measurements. In an example, IMD 10 may be configured to measure intra-vascular impedance and transmit the intra-vascular impedance to an external device 12 or store the intra-vascular impedance measurements locally to IMD 10.

In examples in which IMD 10 monitors the electrical activity of the heart, IMD 10 may sense electrical signals attendant to the depolarization and repolarization of the heart of patient 4 via electrodes.

In some examples, processing circuitry, e.g., of IMD 10, external device 12 or HMS 8, may implement one or more algorithms configured to manipulate data received from sensors of IMD 10. In some examples, the algorithms may include one or more machine learning models configured to accept one or more physiological parameters of patient 4 as input and output a SDB index and/or a risk of HF. For example, other physiological parameters may include a heart rate of the patient, an activity of the patient, a posture of the patient, a blood pressure of the patient, a body fat percentage of the patient, an electrocardiogram of the patient, a tissue impedance of the patient, and an intracardiac electrogram of the patient. In some examples, the processing circuitry determines a number of diagnostic evidence levels based on one or more physiological parameter values, determines a risk of heart failure event based on application of the diagnostic evidence levels to a Bayesian Belief Network, e.g., as described in commonly-assigned U.S. Pat. No. 10,542,887 to Sarkar et al., the entire content of which is incorporated herein by reference. In some such examples, one of the physiological parameters may be any breathing or SDB metric described herein.

System 2 may sense one or more physiological parameters of patient 4 over time and process physiological parameter data to identify episodes and severity of SDB, as well as determine a risk of HF of patient 4. For example, system 2 may receive one or more subcutaneous tissue impedance signals from the electrodes of IMD 10 and, via processing circuitry, determine a waveform corresponding to a breathing pattern of patient 4, e.g., an impedance waveform. The processing circuitry may be processing circuitry of IMD 10, external device 12, and/or other computing devices of system 2. The impedance waveform may include a plurality of tissue impedance values accumulated over a period of time. In some examples, system 2 may continuously analyze sensor signals. For example, the impedance values may be stored in a buffer of impedance values, where the buffer stores impedance values measured over a most recent time period for analysis by system 2 to form the impedance waveform. In some examples, the impedance waveform includes the last ten seconds, two minutes, or any other time period of impedance values.

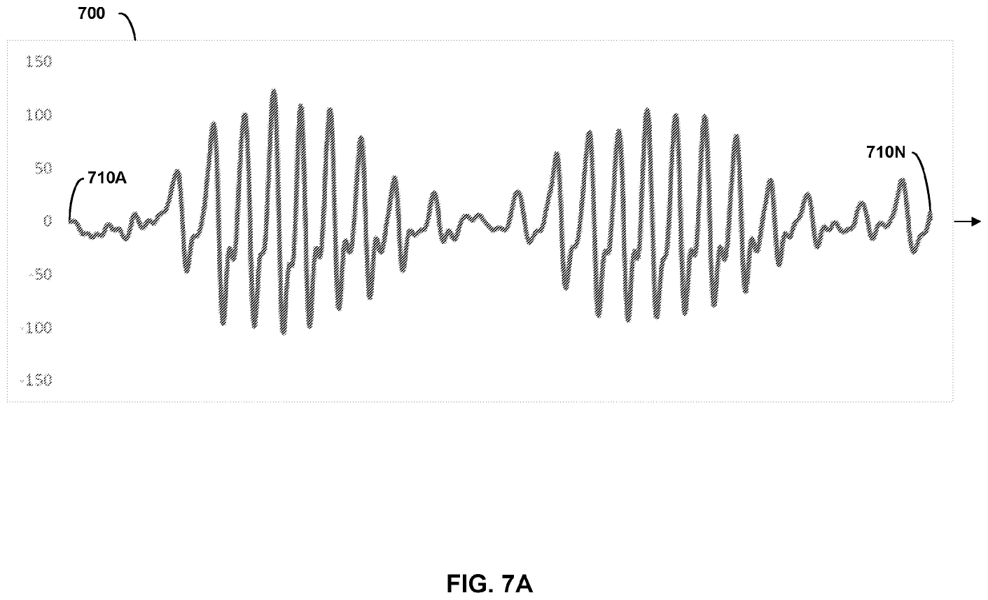

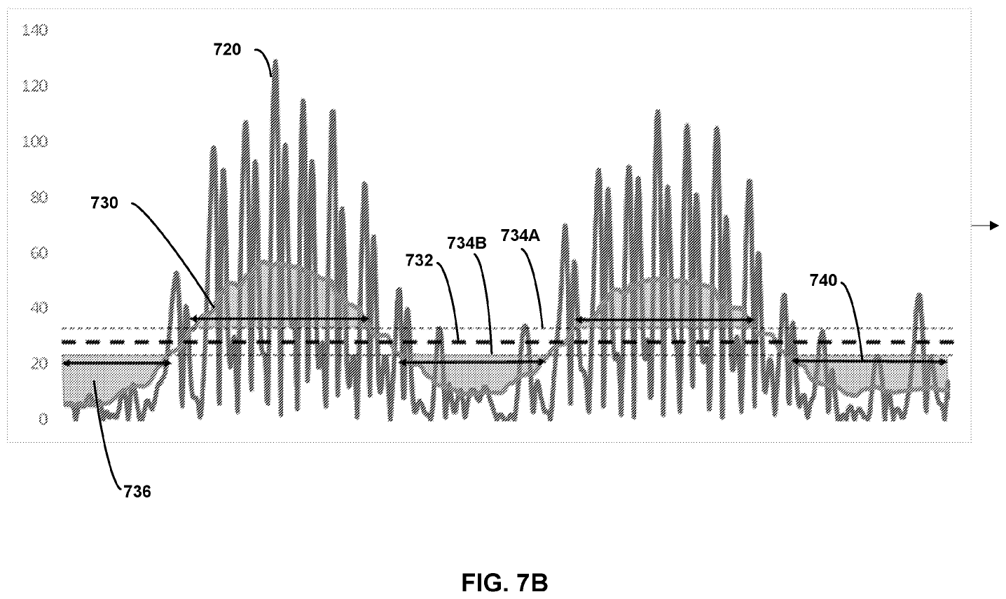

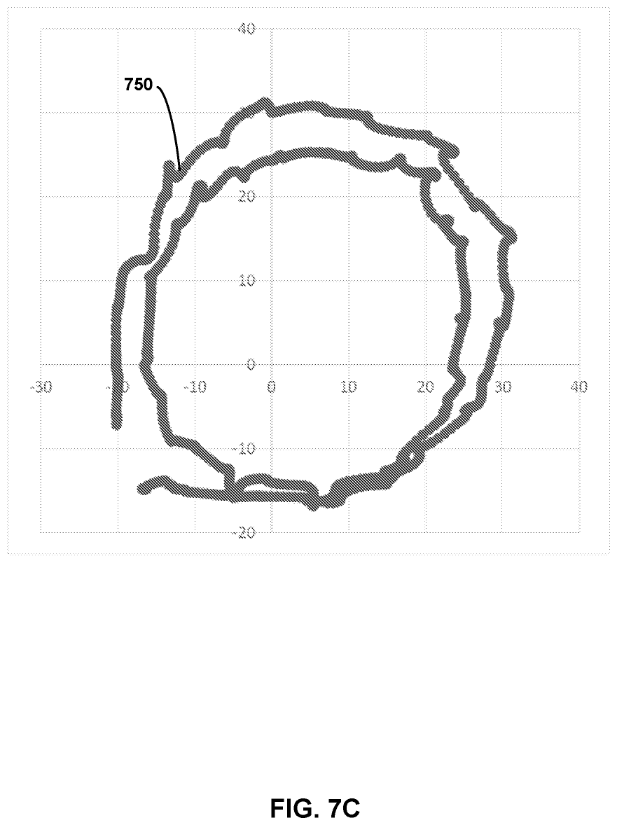

Processing circuitry of system 2 may also determine an envelope signal based on the waveform corresponding to the breathing pattern of the patient, as discussed in greater detail with respect to FIG. 7B. Based at least in part on the envelope signal, system 2 may detect SDB episodes and determine a SDB index. In some examples, the SDB index may be a measure of the quantity of type of patient 4's SDB episodes. For example, the SDB index may be a number (e.g., a number of episodes, a rate of episodes, a number between 1 and 10, or between 1 and 100), where the smaller the number is the more normal patient 4's breathing is, and the higher the number, the more abnormal the breathing is. In some examples, the SDB index may be a classification of patient 4's breathing. For example the SDB index may include terms referencing the type of breathing detected (e.g., “deep breathing”, “shallow breathing”, “normal breathing”, “apnea”, “Cheyne-Stokes breathing”) or numbers correlated to those terms or breathing types.

Processing circuitry of system 2 may also determine a heart condition status of patient 4 based at least in part on the SDB index. In some examples, the heart condition status may represent a risk of HF. For example, the heart condition status may be a number (e.g., between 1 and 100), representing a percentage risk that patient 4 will experience HF. In some examples, the heart condition status may be a string classification (e.g., “at risk of HF”, “not at risk of HF”, “high risk of HF event”) or numbers correlated to those classifications in memory.

In some examples, one or more of IMD 10, external device 12, HMS 8, and another device of system 2 includes a memory in communication with the processing circuitry of system 2. In some examples, the processing circuitry may be configured to determine if the SDB index exceeds a threshold. For example, in examples where the SDB index is represented by a number (e.g., one through one hundred), processing circuitry may determine if the SDB index exceeds a value of sixty. In response to determining that the SDB index exceeds a threshold, the processing circuitry may be configured to save one or more segments of the waveform from which the SDB index was determined to the memory. In examples where the SDB index is a classification category (e.g., “high probability of apnea”), processing circuitry may be configured to save the one or more segments of the waveform in response to the SDB index falling into one or more particular classification categories. A physician or user of external device 12 may be able to access the memory and the saved waveform for further analysis or diagnosis.

In some examples, the processing circuitry may be configured to determine if patient 4 has experienced an SDB episode. For example, system 2 may determine that patient 4 experienced an SDB episode based on the envelope signal, as described below in greater detail with reference to FIG. 7B. In response to determining that patient 4 has experienced an SDB episode, the processing circuitry may save the waveform containing evidence of the SDB episode to a database in memory. A physician or user of external device 12 may be able to access the memory and the saved waveform for further analysis or diagnosis.

In some examples, the processing circuitry may be configured to determine the heart condition status of patient 4, e.g., a risk of HF, based at least in part on the SDB index. The processing circuitry may be configured to determine if the heart condition status exceeds a threshold. For example, in examples where the heart condition status is represented by a number (e.g., one through one hundred as a percent risk of HF), processing circuitry may determine if the heart condition status exceeds a value of sixty. In response to determining that the heart condition status exceeds a threshold, the processing circuitry may be configured to save one or more segments of the waveform from which the heart condition status was determined to the memory. In examples where the heart condition status is a classification category (e.g., “high risk of HF”), processing circuitry may be configured to save the one or more segments of the waveform in response to the heart condition status falling into one or more particular classification categories. A physician or user of external device 12 may be able to access the memory and the saved waveform for further analysis or diagnosis.

The processing circuitry may also be configured to generate an alert (e.g., a notification, a status indicator, an alarm, etc.). For example, processing circuitry may generate an alert in response to one or more of the SDB index exceeding a threshold, detection of an SDB episode in patient 4, and/or a heart condition status exceeding a threshold. In some examples, where the SDB index and/or the heart condition status are represented by a classification category (e.g., “high likelihood of apnea”, “high risk of HF event”), processing circuitry may be configured to generate an alert in response to the SDB index and/or the heart condition status falling into one or more classifications. In a non-limiting example, the alert may include text or graphics information that communicates the waveform, SDB index, SDB episode, heart condition status, or other status of the patient.

In some examples, IMD 10 may transmit the alert to one or more other computing devices (e.g. external device 12 or other computing devices via HMS 8). In some examples where a computing device other than IMD 10 determines the SDB index, SDB episode, and/or heart condition status, that computing device may simply generate the alert or may further transmit the alert to another device of system 2. In some examples, a computing device may transmit an alert to IMD 10 that instructs IMD 10 to take some action in response to the alert. The alert may indicate one or more breathing or heart condition statuses. For example, an alert may trigger for high risk of HF and detection of an SDB episode, and/or a different alert may trigger indicating that patient 4 had a SDB index of “high likelihood of apnea.” In some examples, the type of alert may correspond to the type of status it communicates. For example, an alert for a “high risk of HF” may include a high pitched sound, an alert for a “medium risk of HF” may include a medium-pitched sound, and an alert for a “low risk of HF” may include a soft, low-pitched sound or no sound at all. The alerts may be communicated directly to patient 4 or to the clinician through a variety of methods including notifications, audible tones, handheld devices and automatic or on-demand telemetry to computerized communication network. In some examples, the alert may include an alarm, such as an audible alarm or visual alarm.

FIG. 2 is a conceptual side-view diagram illustrating an example IMD 10 medical system 2 of FIG. 1 in a subcutaneous space 22, in accordance with one or more techniques of this disclosure. FIG. 2 also depicts an example configuration of IMD 10. The conceptual side-view diagram illustrates a muscle layer 20 and a skin layer 18. The region between muscle layer 20 and skin layer 18 includes subcutaneous space 22. Subcutaneous space 22 includes blood vessels 24, such as capillaries, arteries, or veins, and interstitial fluid in the interstitium 28 of subcutaneous space 22. Subcutaneous space 22 has interstitial fluid that is commonly found between skin layer 18 and muscle layer 20. Subcutaneous space 22 may include interstitial fluid that surrounds blood vessels 24. For example, interstitial fluid surrounds capillaries and allows the passing of capillary elements (e.g., nutrients) between the different layers of a body through interstitium 28.

In some examples, IMD 10 may sense impedance changes with respect to interstitial fluid corresponding to a breathing pattern of a patient. In another example, IMD 10 may sense impedance changes with respect to extravascular fluid and other conductive tissues proximate to electrodes 16 corresponding to the breathing pattern of the patient. In any event, IMD 10 may track shifts or changes in impedances of these layers, regardless of which conductive tissue layer and/or type of fluid.

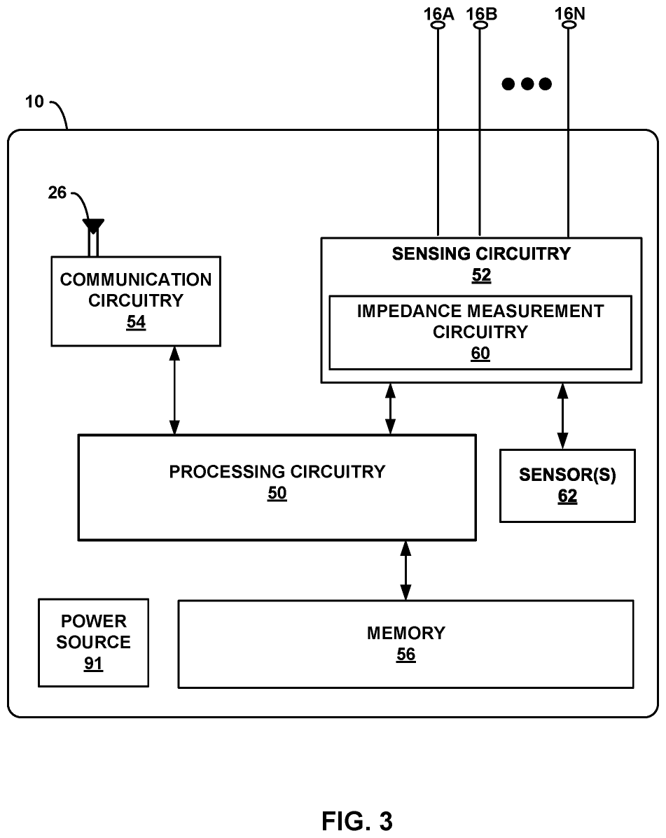

In the example shown in FIG. 2, IMD 10 may include a leadless, subcutaneously-implantable monitoring device having a housing 15 and an insulative cover 76. Electrodes 16A-16N (collectively, “electrodes 16”) may be formed or placed on an outer surface of cover 76. Although the illustrated example includes three electrodes 16, IMDs including or coupled to more or less than three electrodes 16 may implement the techniques of this disclosure in some examples. For example, electrode 16N or additional electrodes may be unnecessary in some instances, e.g., in which housing 15 is conductive and acts as an electrode of IMD 10. Circuitries 50-62, described below with respect to FIG. 3, may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26, circuitries 50-62, and accelerometer 30, and protect the antenna and circuitries from fluids such as interstitial fluids or other bodily fluids.

IMD 10 may also include an accelerometer 30. In some examples, accelerometer 30 may be one or more of circuitries 50-62. In some examples, accelerometer 30 may include one or more accelerometers for three-axis motion detection. Although depicted as part of IMD 10, in some examples accelerometer 30 may be part of a device external to the patient but attached to the patient's body and configured to detect patient motion. Processing circuitry of IMD 10 may be configured to collect an accelerometer signal from the accelerometer, where the accelerometer signal is indicative of patient movement. For example, processing circuitry may determine that changes in a y-axis accelerometer signal indicate vertical patient motion. Impedance measurements corresponding to breathing patterns of the patient may be inaccurate when the patient is otherwise moving. Therefore, processing circuitry may determine a time period in which the patient is moving based on the accelerometer signal. For example, large changes in a y-axis accelerometer signal may indicate that the patient is stretching in their sleep or performing a ballet sauté, and the processing circuitry may determine that the patient is moving. In some examples, a period of large changes in the z-axis accelerometer signal may indicate that the patient is rolling in their sleep or performing a pirouette, and the processing circuitry may determine that the patient is moving. The processing circuitry may determine a time period in which the patient is moving, for example via an internal clock. The processing circuitry may determine an SDB index of the patient based on an envelope signal over a time period that does not overlap with the time period in which the patient is moving. In this way, the system may ensure the most accurate impedance measurements for determination of the SDB index.

IMD 10 can face outward toward skin layer 18, inward toward muscle layer 20, or perpendicular in any direction (e.g., left, right, into the page of FIG. 2, out of the page of FIG. 2). For example, IMD 10 may be oriented to face outward toward the skin, as shown in FIG. 2. In some examples, IMD 10 may be oriented vertically relative to the skin layer 18 and muscle layer 20 such that the electrodes face to the left of the page of FIG. 2 or to the right of the page of FIG. 2. In other examples, IMD 10 may be oriented diagonally or horizontally (as shown in FIG. 2). Although shown with a particular orientation in FIG. 2, a person of skill in art would understand that IMD 10 can have various orientations and that the orientation in FIG. 2 is for illustrative purposes. Similarly, a person of skill in the art would understand that IMD 10 may be positioned closer to muscle layer 20 than to an outer layer of skin layer 18 (e.g., dermis layer or epidermis layer), whereas at other times, IMD 10 may be closer to an outer layer of skin layer 18 (e.g., dermis layer or epidermis layer).

IMD 10 may also be any shape (e.g., circular, square, rectangular, trapezoidal, etc.). For example, as shown in FIG. 2, IMD 10 has a particular shape having rounded edges across the housing 15. In addition, electrodes 16 may be positioned around the perimeter of the shape or around a partial perimeter of the shape (as shown in FIG. 2).

In some instances, the configuration of electrodes 16 is selected so as to maximize the accuracy of the impedance measurements based on a relative location of circuitries 50-62. The location of circuitries 50-62 may be based on form factor and other considerations (charging, electromagnetic noise reduction, etc.) such that electrodes 16 may be positioned as an indirect effect of the selected configuration of circuitries 50-62. In other examples, electrodes may be positioned irrespective of the configuration of circuitries 50-62 and instead, may be based on other design considerations such as the relative locations of blood vessels 24 within an implant region. For example, electrodes 16 may be positioned so as to face a capillary of interest or group of capillaries that may be utilized to provide an even more accurate depiction of impedance changes over time. For instance, IMD 10 may determine that an optimal impedance reading is available nearer certain blood vessels 24 compared to other blood vessels 24. IMD 10 may have allow for self-repositioning to take advantage of the optimal reading, for example, through remote control operations, magnetic repositioning, etc. For example, IMD 10 may receive a remote-control signal or magnetic impulse causing IMD 10 to rotate in a desired direction (clockwise, counterclockwise, etc.) in order to achieve such optimal readings.

IMD 10 may be configured to float within interstitium 28 or may be fixed in place, for example, using lead wires as a tether allowing controlled degrees of freedom depending on the lead wire configuration. For example, lead wires having more slack may allow IMD 10 more degrees of freedom to float within interstitium 28.

In some examples, at least one of electrodes 16 of IMD 10 may disposed within another layer, such as muscle layer 20 or skin layer 18. In other examples, electrodes 16 may be disposed all within a single layer, such as subcutaneous space 22. In any event, at least one of electrodes 16 will contact interstitial fluid in subcutaneous space 22, whereas other electrodes 16 may not contact interstitial fluid. In other examples, each of electrodes 16 or at least two of electrodes 16 will contact interstitial fluid in subcutaneous space 22. In addition, at least two of the electrodes 16 may be positioned approximately 3 cm-5 cm apart, such as at 4 cm apart. In another example, some or all of electrodes 16 may be positioned closer or farther away than 4 cm.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (e.g., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

FIG. 3 is a functional block diagram illustrating an example configuration of IMD 10 of FIGS. 1 and 2 in accordance with one or more techniques described herein. In the illustrated example, IMD 10 includes electrodes 16, antenna 26, processing circuitry 50, sensing circuitry 52, impedance measurement circuitry 60, communication circuitry 54, memory 56, sensors 62, and power source 91.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16, e.g., to select the electrodes 16 and polarity, referred to as the sensing vector, used to sense impedance and/or cardiac signals, as controlled by processing circuitry 50. Sensing circuitry 52 may sense signals from electrodes 16, e.g., to produce a cardiac EGM or subcutaneous electrocardiogram (ECG), in order to facilitate monitoring the electrical activity of the heart. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

In some examples, processing circuitry 50 may use switching circuitry to select, e.g., via a data/address bus, which of the available electrodes are to be used to obtain impedance measurements of interstitial fluid. The switching circuitry may include a switch array, switch matrix, multiplexer, transistor array, microelectromechanical switches, or any other type of switching device suitable to selectively couple sensing circuitry 52 to selected electrodes. In some examples, sensing circuitry 52 includes one or more sensing channels, each of which may include an amplifier. In response to the signals from processing circuitry 50, switching circuitry 58 may couple the outputs from the selected electrodes to one of the sensing channels.

In some examples, one or more channels of sensing circuitry 52 may include R-wave amplifiers that receive signals from electrodes 16. In some examples, the R-wave amplifiers may take the form of an automatic gain-controlled amplifier that provides an adjustable sensing threshold as a function of the measured R-wave amplitude. In addition, in some examples, one or more channels of sensing circuitry 52 may include a P-wave amplifier that receives signals from electrodes 16. Sensing circuitry may use the received signals for pacing and sensing in the heart of patient 4. In some examples, the P-wave amplifier may take the form of an automatic gain-controlled amplifier that provides an adjustable sensing threshold as a function of the measured P-wave amplitude. Other amplifiers may also be used. In some examples, sensing circuitry 52 includes a channel that includes an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in memory 56. Processing circuitry 50 may employ digital signal analysis techniques to characterize the digitized signals stored in memory 56 to detect and classify cardiac arrhythmias from the digitized electrical signals.

Sensing circuitry 52 includes impedance measurement circuitry 60. Processing circuitry 50 may control impedance circuitry 60 to periodically or continually measure an electrical parameter to determine an impedance, such as a subcutaneous impedance indicative of breathing patterns of a patient. For a subcutaneous impedance measurement, processing circuitry 50 may control impedance measurement circuitry 60 to deliver an electrical signal between selected electrodes 16 and measure a current or voltage amplitude of the signal. Processing circuitry 50 may select any combination of electrodes 16, e.g., by using switching circuitry and sensing circuitry 52. Impedance measurement circuitry 60 includes sample and hold circuitry or other suitable circuitry for measuring resulting current and/or voltage amplitudes. Processing circuitry 50 determines an impedance value from the amplitude value(s) received from impedance measurement circuitry 60. In some examples, processing circuitry 50 may include switching circuitry to switch between measurements of ECG and impedance measurements across the same electrodes 16. For example, the switching circuitry may use multiplexing to switch between measurements, such that processing circuitry 50 may utilize electrodes 16 to perform various measurements (e.g., impedance, ECG, etc.). In such examples, processing circuitry 50 may receive a plurality of signals using electrodes 16, where the signals include at least one electrocardiogram (ECG) and/or one or more subcutaneous tissue impedance signals.

In some examples, IMD 10 may include measurement circuitry having an amplifier design configured to switch in real-time and continuously between impedance value measurements and other physiological parameter measurements, such as ECG. In addition, IMD 10 may enable impedance measurement circuitry 60 for short periods of time in order to conserve power. In some examples, IMD 10 may include an accelerometer configured to detect patient motion. In order to conserve power, IMD 10 may not enable impedance measurement circuitry while detecting that the patient is in motion via the accelerometer. In one example, IMD 10 may use an amplifier circuit, such as a chopper amplifier, according to certain techniques described in U.S. application Ser. No. 12/872,552 by Denison et al., entitled “CHOPPER-STABILIZED INSTRUMENTATION AMPLIFIER FOR IMPEDANCE MEASUREMENT,” filed on Aug. 31, 2010, incorporated herein by reference in its entirety.

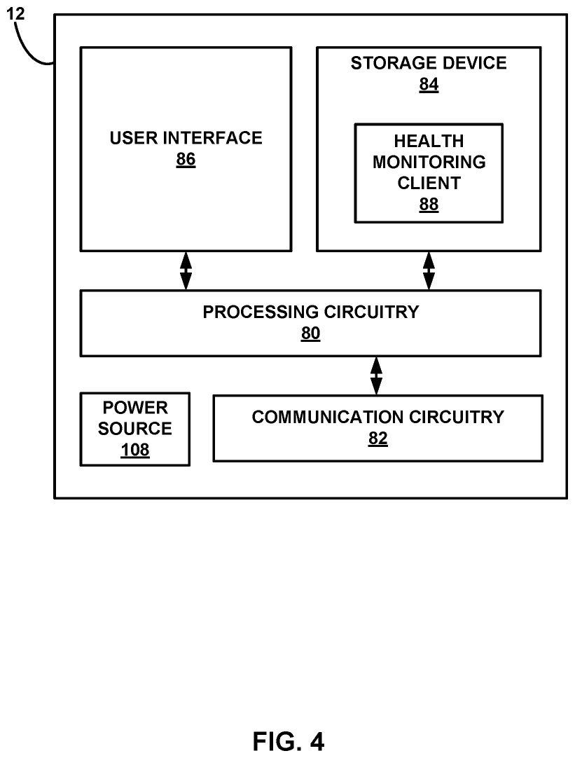

Because either IMD 10 or external device 12 may be configured to include sensing circuitry 52, impedance measurement circuitry 60 may be implemented in one or more processors, such as processing circuitry 50 of IMD 10 or processing circuitry 80 of external device 12 as shown in FIG. 4. Impedance measurement circuitry 60 is, in the example described with reference to FIG. 3, shown in conjunction with sensing circuitry 52 of IMD 10. Similar to processing circuitry 50 and other circuitry described herein, impedance measurement circuitry 60 may be embodied as one or more hardware modules, software modules, firmware modules, or any combination thereof. Impedance measurement circuitry 60 may analyze impedance measurement data continuously or on a periodic basis to build impedance waveforms indicative of patient breathing patterns.

In some examples, impedance measurement circuitry 60 may measure impedance values in response to receiving a signal from one or more other medical devices (e.g., via communication circuitry 54). In some examples, the one or more other medical devices may include a sensor device, such as an activity sensor, heart rate sensor, a wearable device worn by patient 4, a temperature sensor, etc. That is, the one or more other medical devices may, in some examples, be external to IMD 10. In such examples, the other medical devices may interface with IMD 10 via communication circuitry 54. In some examples, impedance measurement circuitry 60 may measure impedance values in response to receiving a signal from processing circuitry 50. For example, in response to one or more quantifications of an ECG of patient 4 satisfying a threshold, processing circuitry 50 may initiate impedance measurement circuitry 60 to measure impedance values.

In some examples, IMD 10 may include the one or more other medical devices, such as by having the other medical devices included within housing 15 or otherwise fixed to an inner or outer portion of IMD 10. For example, the other medical device may include one or more of sensors (e.g., an accelerometer) affixed to an inner or outer portion of IMD 10. In some examples, processing circuitry 50 may receive one or more signals from one or more medical devices that trigger processing circuitry 50 to control impedance measurement circuitry 60 to perform impedance measurements.

For example, impedance measurement circuitry 60 may determine a received signal includes a trigger that causes impedance measurement circuitry 60 to measure one or more impedance values using electrodes 16. In a non-limiting example, impedance measurement circuitry 60 may receive signals (e.g., from an accelerometer) indicating when patient 4 has low activity. In response to receiving the signal indicating an activity level, impedance measurement circuitry 60 may measure one or more impedance values using electrodes 16. In another example, impedance measurement circuitry 60 may receive signals indicating when patient 4 has lower or higher heart rate compared to that of a heart rate threshold, etc. In some examples, impedance measurement circuitry 60 may receive signals from the ECG processing circuitry indicating periodic changes in the R-wave amplitude, suggestive of SDB. In any event, impedance measurement circuitry 60 may determine whether the received signals includes triggering information that communicates to impedance measurement circuitry 60 that impedance measurement circuitry 60 is to perform physiological parameter measurements using electrodes 16.

In some examples, processing circuitry 50 may determine whether a combination of one or more signals received from one or more transmitting devices contains triggering information. For example, processing circuitry 50 may receive a signal indicating a low activity level, a signal indicating a low heart rate, and a signal indicating a low temperature, where each is below a threshold (e.g., if processing circuitry 50 determines that there is greater than a threshold likelihood that the patient is sleeping). In response to each of the received signals being below a threshold, processing circuitry 50 may cause impedance measurement circuitry 60 to measure one or more impedance values using electrodes 16. In some examples, processing circuitry 50 may additionally use timing information. For example, processing circuitry 50 may start a timer based on the triggering information. In some examples, processing circuitry 50 may cause impedance measurement circuitry 60 to measure impedance values in accordance with a timing constraint (e.g., only perform measurements at night) following a triggering event, regardless of when the triggering event occurred during the day. In any event, processing circuitry 50 may cause IMD 10 to determine one or more tissue impedance values in response to the triggering event, such as in response to receiving a signal from a sensor device, where in some instances, IMD 10 may include the sensor device or the sensor device may be independent of IMD 10.

In some examples, impedance measurement circuitry 60 may continuously measure impedance values over a time period, such at night or when physiological parameter data indicates that the patient is asleep. The impedance values may be stored in a buffer of impedance values, where the buffer stores impedance values measured over a most recent time period for analysis by processing circuitry 50 to form the impedance waveform. In some examples, the impedance waveform includes the last ten seconds, two minutes, or any other time period of impedance values.

In some examples, impedance measurement circuitry 60 may be configured to sample impedance measurements at a particular sampling rate. In such examples, impedance measurement circuitry 60 may be configured to perform downsampling of the received impedance measurements. For example, impedance measurement circuitry 60 may perform downsampling in order to decrease the throughput rate or to decrease the amount of data transmitted to processing circuitry 50. This may be particularly advantageous where impedance measurement circuitry 60 has a high sampling rate when active.

In some examples, processing circuitry 50 may perform impedance measurements by causing impedance measurement circuitry 60 (e.g., via switching circuitry) to deliver a voltage pulse between at least two electrodes 16 and examining resulting current amplitude value measured by impedance measurement circuitry 60. In some examples, switching circuitry may deliver signals that deliver stimulation therapy to the heart of patient 4. In other examples, these signals may be delivered during a refractory period, in which case they may not stimulate the heart of patient 4.

In other examples, processing circuitry 50 may perform an impedance measurements by causing impedance measurement circuitry 60 to deliver a current pulse across at least two selected electrodes 16. Impedance measurement circuitry 60 holds a measured voltage amplitude value. Processing circuitry 50 determines an impedance value based upon the amplitude of the current pulse and the amplitude of the resulting voltage that is measured by impedance measurement circuitry 60. IMD 10 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue, subcutaneous tissue, or muscle tissue.

In certain cases, IMD 10 may measure subcutaneous impedance values that include both a resistive component and a reactive component (e.g., X, XL, XC), such as in an impedance triangle. In such cases, IMD 10 may measure subcutaneous impedance during delivery of a sinusoidal or other time varying signal by impedance measurement circuitry 60, for example. Thus, as used herein, the term “impedance” is used in a broad sense to indicate any collected, measured, and/or calculated value that may include one or both of resistive and reactive components. In some examples, subcutaneous tissue impedance values are derived from subcutaneous tissue impedance signals received from electrodes 16.

In the example illustrated in FIG. 3, processing circuitry 50 is capable of performing the various techniques described throughout the disclosure. To avoid confusion, processing circuitry 50 is described as performing the various impedance processing techniques proscribed to IMD 10, but it should be understood that these techniques may also be performed by other processing circuitry (e.g., processing circuitry 80 of external device 12, etc.). In various examples, processing circuitry 50 may perform one, all, or any combination of the plurality of impedance waveform analysis techniques discussed in greater detail below.

In some examples, processing circuitry 50 may be configured to determine if the SDB index exceeds a threshold. For example, in examples where the SDB index is represented by a number (e.g., one through one hundred), processing circuitry 50 may determine if the SDB index exceeds a value of sixty. This number is presented for example only, and the SDB index threshold may be any value which may be programmed in memory. In response to determining that the SDB index exceeds a threshold, processing circuitry 50 may be configured to save one or more segments of the impedance waveform from which the SDB index was determined to memory 56. In examples where the SDB index is a classification category (e.g., “high likelihood of apnea”), processing circuitry 50 may be configured to save the one or more segments of the impedance waveform in response to the SDB index falling into one or more particular classification categories. A physician or user of an external device may be able to access memory 56 and the saved impedance waveform for further analysis or diagnosis.

In some examples, processing circuitry 50 may be configured to determine if patient 4 has experienced an SDB episode. For example, system 2 may determine that patient 4 experienced an SDB episode based on the envelope signal, as described below in greater detail with reference to FIG. 7B. In some examples, processing circuitry 50 may determine that an SDB episode has occurred in patient 4 in response to the determined SDB index exceeding a threshold. In response to determining that patient 4 has experienced an SDB episode, processing circuitry 50 may save the impedance waveform containing evidence of the SDB episode to a database in memory 56. A physician or user of external device 12 may be able to access memory 56 and the saved impedance waveform for further analysis or diagnosis.

In some examples, processing circuitry 50 may be configured to determine the heart condition status of patient 4 based at least in part on the SDB index. Processing circuitry 50 may be configured to determine if the heart condition status exceeds a threshold. For example, in examples where the heart condition status is represented by a number (e.g., one through one hundred as a percent risk of HF), processing circuitry 50 may determine if the heart condition status exceeds a value of sixty. This threshold is chosen for example only, and the threshold may be any value, for example a value programmed in memory by a physician In response to determining that the heart condition status exceeds a threshold, processing circuitry 50 may be configured to save one or more segments of the impedance waveform from which the heart condition status was determined to memory 56. In examples where the heart condition status is a classification category (e.g., “high risk of HF”), processing circuitry 50 may be configured to save the one or more segments of the impedance waveform in response to the heart condition status falling into one or more particular classification categories. A physician or user of external device 12 may be able to access the memory and the saved impedance waveform for further analysis or diagnosis.

Processing circuitry 50 may also be configured to generate an alert (e.g., a notification, a status indicator, an alarm, etc.). For example, processing circuitry 50 may generate an alert in response to one or more of the SDB index exceeding a threshold, detection of an SDB episode in patient 4, and/or a heart condition status exceeding a threshold. In some examples, where the SDB index and/or the heart condition status are represented by a classification category (e.g., “high likelihood of apnea”, “high risk of HF event”), processing circuitry 50 may be configured to generate an alert in response to the SDB index and/or the heart condition status falling into one or more classifications. In some examples, IMD 10 may provide an audible or tactile alert in the form of a beeping noise or a vibrational pattern. Alternatively, IMD 10 may send an alert signal to external device 12 that causes external device 12 to provide an alert to patient 4. External device 12 may provide an audible, visual, or tactile alert to patient 4. Once patient 4 is alerted, patient 4 may then seek medical attention, e.g., by checking into a hospital or clinic. The alerts may be separated into various degrees of seriousness as indicated by an impedance score.

Sensing circuitry 52 may also provide one or more impedance signals to processing circuitry 50 for analysis, e.g., for analysis to determine an impedance waveform, an envelope signal, an SDB index, and/or a heart condition status according to the techniques of this disclosure. In some examples, processing circuitry 50 may store the impedance waveform, envelope signal, SDB index, and/or heart rate condition status in memory 56. Processing circuitry 50 of IMD 10, and/or processing circuitry of another device that retrieves data from IMD 10, may analyze the data stored in memory 56 to determine a cardiac condition of patient 4 according to the techniques of this disclosure. In some examples, IMD 10 may store the impedance waveform in memory 56, and processing circuitry of another device may retrieve the impedance waveform from memory 56 via communication circuitry 54 to analyze the impedance waveform. Exporting the impedance waveform to another device for subsequent data analysis may preserve a battery life of IMD 10.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network.

Antenna 26 and communication circuitry 54 may be configured to transmit and/or receive signals via inductive coupling, electromagnetic coupling, NFC technologies, RF communication, Bluetooth®, Wi-Fi™, or other proprietary or non-proprietary wireless communication schemes. In some examples, processing circuitry 50 may provide data to be uplinked to external device 12 via communication circuitry 54 and control signals using an address/data bus. In another example, communication circuitry 54 may provide received data to processing circuitry 50 via a multiplexer.

In some examples, processing circuitry 50 may send impedance data to external device 12 via communication circuitry 54. For example, IMD 10 may send external device 12 collected impedance measurements which are then analyzed by external device 12. In such examples, external device 12 performs the described processing techniques. Alternatively, IMD 10 may perform the processing techniques and transmit the processed impedance data to external device 12 for reporting purposes, e.g., for providing an alert to patient 4 or another user.

In some examples, memory 56 may be a storage device that includes computer-readable instructions that, when executed by processing circuitry 50, cause IMD 10 and processing circuitry 50 to perform various functions attributed to IMD 10 and processing circuitry 50 herein. Memory 56 may include any volatile, non-volatile, magnetic, optical, or electrical media. For example, memory 56 may include random access memory (RAM), read-only memory (ROM), non-volatile RAM (NVRAM), electrically-erasable programmable ROM (EEPROM), erasable programmable ROM (EPROM), flash memory, or any other digital media. Memory 56 may store, as examples, programmed values for one or more operational parameters of IMD 10 and/or data collected by IMD 10 for transmission to another device using communication circuitry 54. Data stored by memory 56 and transmitted by communication circuitry 54 to one or more other devices may include impedance values and/or digitized cardiac EGMs, as examples.

The various components of IMD 10 are coupled to power source 91, which may include a rechargeable or non-rechargeable battery. A non-rechargeable battery may be capable of holding a charge for several years, while a rechargeable battery may be inductively charged from an external device, such as external device 12, on a daily, weekly, or annual basis, for example.

During normal operation, IMD 10 may collect only minimal data from patient tissue, or only minimal physiological parameters from the patient using sensors of IMD 10 in order to conserve power of power source 91. IMD 10 may also only operate intermittently, e.g., 20 minutes at a time once a day. In some examples, a user of external device 12 may adjust settings to IMD 10 to control a tradeoff between data collection and battery longevity. For example, a user may adjust the settings of IMD 10 to change the type of data collected by IMD 10 (e.g., when to start or stop impedance measurements, accelerometer measurements, ECG measurements, or measurements of any other sensors of IMD 10), to change a length of time data is collected by IMD 10 (e.g., impedance measurements may only be collected for 30 minute increments or only a certain number of times per week, etc.), and/or to change the amount of data processing performed by processors 50 of IMD 10 (e.g., a breathing waveform may be sent to an external device for signal processing).

In some examples, IMD 10 may determine an SDB index for patient 4 indicating a high likelihood of sleep apnea. In some examples IMD 10 may determine that patient 4 has experienced a high number of SDB episodes within a time period. In these examples, in order to conserve battery power and after sending an alert indicating the SDB index or number of SDB episodes, IMD 10 may reduce the amount of time sensing signals corresponding to breathing patterns.

In some examples, IMD 10 may prioritize activation of one or more sensors based on batter power. For example, IMD 10 may activate ECG sensors to sense signals corresponding to a breathing pattern of patient 4 (e.g., by changes in R-wave amplitude), and may disable impedance measurement circuitry to conserve battery power. In some examples, IMD 10 may active impedance measurement circuitry in response to identifying one or more SDB episodes based on ECG sensors. The impedance measurement circuitry may operate for a time period sufficient to confirm or disconfirm ECG identifications of SDB episodes before deactivating to conserve battery power.

FIG. 4 is a functional block diagram illustrating an example configuration of external device 12 of FIG. 1, in accordance with one or more techniques of this disclosure. In some examples, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC technologies, RF communication, Bluetooth®, Wi-Fi™, or other wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution. Storage device 84 may also store historical impedance data, timing information (e.g., number and durations of SDB episodes, impedance waveforms, envelope signals, SDB indexes, HF condition statuses, etc.).

Data exchanged between external device 12 and IMD 10 may include operational parameters (e.g., resolution parameters). External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., impedance waveform, envelope signal, SDB index, and/or heart condition statuses) to external device 12. In turn, external device 12 may receive the collected data from IMD 10 and store the collected data in storage device 84. Processing circuitry 80 may implement any of the techniques described herein to analyze impedance values received from IMD 10, e.g., to determine envelope signals, SDB indexes, etc. Using the impedance analysis techniques disclosed herein, processing circuitry 80 may determine a heart condition status of patient 4 and/or generate an alert based on the heart condition status.

A user, such as a clinician or patient 4, may interact with external device 12 through user interface 86. User interface 86 includes a display (not shown), such as an LCD or an LED display or other type of screen, with which processing circuitry 80 may present information related to IMD 10, e.g., cardiac EGMs, indications of detections of impedance changes, impedance waveforms, envelope signals, and quantifications of SDB episodes. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

Power source 108 delivers operating power to the components of external device 12. Power source 108 may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within external device 12. In other embodiments, traditional batteries (e.g., nickel cadmium or lithium ion batteries) may be used. In addition, external device 12 may be directly coupled to an alternating current outlet to power external device 12. Power source 108 may include circuitry to monitor power remaining within a battery. In this manner, user interface 86 may provide a current battery level indicator or low battery level indicator when the battery needs to be replaced or recharged. In some cases, power source 108 may be capable of estimating the remaining time of operation using the current battery.

Storage device 84 may also include an HMS client 88 of HMS 8. HMS client 88 may receive and process data from IMD 10, and may transmit data to HMS 8. Although shown as implemented in storage device 84, in some examples HMS client 88 may additionally or alternatively be implemented in other devices of the system (e.g., IMD 10), or in the cloud. In some examples, HMS client 88 may include one or more algorithms configured to manipulate data received from sensors of IMD 10. In some examples, HMS client 88 may include one or more machine learning models configured to accept one or more physiological parameters of patient 4 as input and output a SDB index and/or a risk of HF.

In some examples, HMS client 88 may (via processing circuitry 80) determine an impedance waveform corresponding to a breathing pattern of the patient based at least in part on one or more subcutaneous tissue impedance signals measured by IMD 10. The impedance waveform may include a plurality of tissue impedance signals, e.g., HMS 8 may build the impedance waveform from a series of the impedance signals measured over a time period by IMD 10. HMS 8 may also determine an envelope signal based on the impedance waveform. In some examples, HMS 8 may detect one or more SDB episodes based on the envelope signal, and may determine an SDB index based on one or more of the envelope signal and a quantification of the one or more SDB episodes.

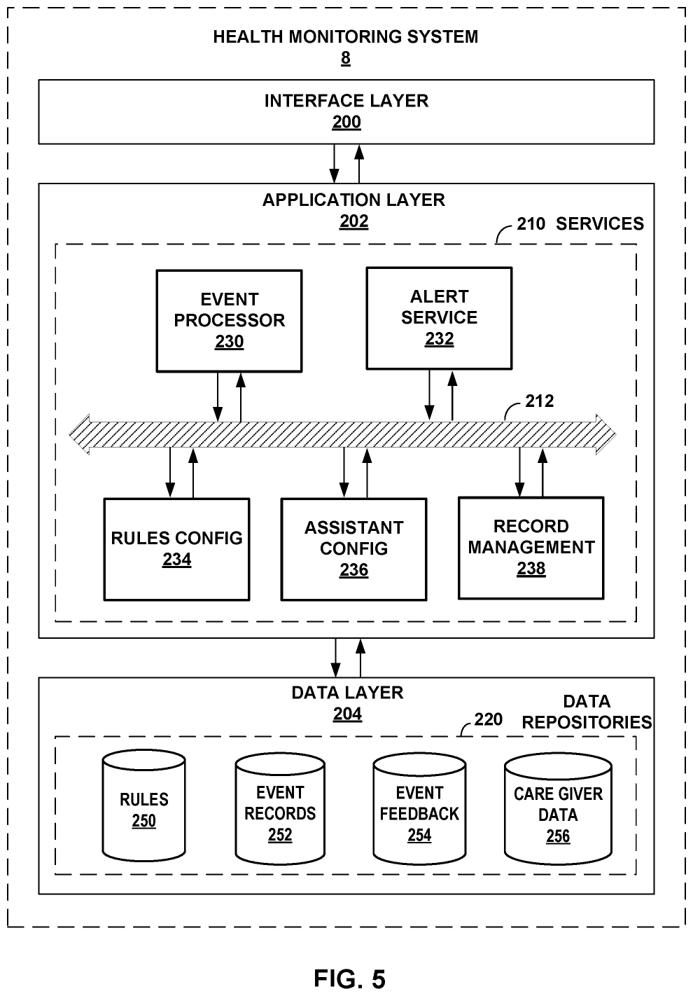

FIG. 5 is a block diagram illustrating an example configuration of health monitoring system 8 that operates in accordance with one or more techniques of the present disclosure. HMS 8 may be implemented in a medical system 2 of FIG. 1, which may include hardware components such as those of IMD 10 and external device 12, e.g., processing circuitry, memory, and communication circuitry, embodied in one or more physical devices. FIG. 5 provides an operating perspective of HMS 8 when hosted as a cloud-based platform. In the example of FIG. 5, components of HMS 8 are arranged according to multiple logical layers that implement the techniques of this disclosure. Each layer may be implemented by one or more modules comprised of hardware, software, or a combination of hardware and software.

Computing devices, such as IMD 10, external device 12, or other computing devices of system 2 of FIG. 1 may operate as clients that communicate with HMS 8 via interface layer 200. The computing devices may execute client software applications, such as desktop application, mobile application, and web applications. Interface layer 200 represents a set of application programming interfaces (API) or protocol interfaces presented and supported by HMS 8 for the client software applications. Interface layer 200 may be implemented with one or more web servers.

As shown in FIG. 5, HMS 8 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model. As illustrated in the example of FIG. 5, services 210 may also include an assistant configuration service 236 for configuring and interacting with an event assistant implemented in external device 12 or other computing devices.

Data layer 204 of HMS 8 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 5, each of services 230-238 is implemented in a modular form within HMS 8. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from IMD 10 and/or external device 12 indicating that IMD 10 detected a potential health event of patient (e.g., an SDB episode) and, in some examples, that the transmitting device confirmed the detection. Event processor service 230 may initiate performance of any of the operations in response to detection of a potential health event ascribed herein to HMS 8, such as communicating with patient 4, a physician, or other care providers, and, in some cases, analyzing data (e.g., to determine an SDB index based on the SDB episode, to determine a heart condition status based at least in part on the SDB index, etc.).

Record management service 238 may store the patient data included in a received alert message within event records 252. Patient data included in a received alert may include physiological parameter data of the patient (e.g., a heart rate, impedance waveform, activity level, EGM, etc.) and/or other data related to or derived from the physiological parameter data (e.g., time of the potential health event, an envelope signal corresponding to a breathing pattern of the patient, etc.). Alert service 232 may package the some or all of the data from the event record, in some cases with additional information as described herein, into one more alert messages for transmission to patient 4 and/or a care provider. Care giver data 256 may store data used by alert service 232 to identify to whom to send alerts based on locations of care givers relative to a location of patient 4 and/or applicability of the care provided by the care givers to the potential health event experienced by patient 4.

In examples in which HMS 8 performs an analysis of the patient data (e.g., determines an SDB index based on a quantification of one or more SDB episodes, determines a heart condition status based on one or more of the SDB index and physiological parameters of the patient), event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Belief Network, Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

HMS 22 may be configured to determine the heart condition status of the patient based on application of the machine learning models to the SDB index and one or more other physiological parameters of the patient. For example, HMS 8 may apply the SDB index and the one or more other physiological parameters of the patient as input to the machine learning model. HMS 8 may determine, as output from the machine learning model, the heart condition status of the patient. In some examples, other physiological parameters may include a heart rate of the patient, an activity of the patient, a posture of the patient, a blood pressure of the patient, a body fat percentage of the patient, a tissue impedance of the patient, an electrocardiogram of the patient, and an intracardiac electrogram of the patient.

In some examples, in addition to rules used by HMS 8 to analyze the patient data, rules 250 maintained by HMS 8 may include rules utilized by external device 12 and rules used by IMD 10. In such examples, rules configuration service 250 may be configured to develop and maintain the rules of external device 12 and/or IMD 10. Rules configuration service 234 may be configured to develop different sets of rules, e.g., different machine learning models, for different cohorts of patients. Rules configuration service 234 may be configured to modify these rules based on event feedback data 254 that indicates whether the determinations of SDB indexes and/or heart condition statuses by IMD 10, external device 12, and/or HMS 8 were accurate. Event feedback 254 may be received from patient 4, e.g., via external device 12, or from a care provider. In some examples, rules configuration service 234 may utilize event records from true and false detections (as indicated by event feedback data 254) and confirmations for supervised machine learning to further train models included as part of rules 250.

FIG. 6 is a block diagram illustrating an example system that includes an access point 90, a network 92, external computing devices, such as a server 94, and one or more other computing devices 100A-100N (collectively, “computing devices 100”). which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 6, access point 90, external device 12, server 94, and computing devices 100 are interconnected and may communicate with each other through network 92. Network 92 may include a local area network, wide area network, or global network, such as the Internet. In some examples, server 94 may be an example of, an example of device configured to implement, or a component of HMS 8 of FIG. 5. The system of FIG. 6 may be implemented, in some aspects, with general network technology and functionality similar to that provided by the Medtronic CareLink® Network.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as impedance value information, impedance scores, and/or cardiac electrograms (EGMs), to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 6 may be implemented with general network technology and functionality, which may be similar to that provided by the Medtronic CareLink® Network.

In some examples, server 94 may monitor impedance, e.g., based on measured impedance information received from IMD 10 and/or external device 12 via network 92, to detect breathing patterns, determine SDB episodes, SDB indexes, and/or heart condition statuses of patient 4 using any of the techniques described herein. Server 94 may provide alerts relating to worsening sleep apnea or worsening HF of patient 4 via network 92 to patient 4 via access point 90, or to one or more clinicians via computing devices 100. In examples such as those described above in which IMD 10 and/or external device 12 monitor the impedance, server 94 may receive an alert from IMD 10 or external device 12 via network 92, and provide alerts to one or more clinicians via computing devices 100. In some examples, server 94 may generate web-pages to provide alerts and information regarding the impedance, and may include a memory to store alerts and diagnostic or physiological parameter information for a plurality of patients. In some examples, server 94 may include one or more virtual machines (e.g., cloud computing devices) configured to perform one or more techniques of the disclosure.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4.

In some examples, instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 to proactively seek medical attention prior to receiving instructions for a medical intervention. In this manner, patient 4 may be empowered to take action, as needed, to address his or her medical status, which may help improve clinical outcomes for patient 4.

In the example illustrated by FIG. 6, server 94 includes a storage device 96, e.g., to store data retrieved from IMD 10, and processing circuitry 98. Although not illustrated in FIG. 6 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to analyze impedance values received from IMD 10, e.g., to determine an SBD index of patient 4 (e.g., worsening sleep apnea) and/or a heart condition status of patient 4 (e.g., worsening HF).

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

FIG. 7A is a graph illustrating an example impedance waveform 700, in accordance with one or more techniques of this disclosure. The medical system may include an IMD with one or more sensors configured to sense one or more physiological parameters of a patient. For example, the IMD may include a plurality of electrodes configured for subcutaneous implantation outside of a thoracic cavity of a patient. The IMD may sense, by the one or more sensors, one or more sensor signals indicative of the one or more physiological parameters of the patient. For example, the IMD may be configured to receive one or more subcutaneous tissue impedance signals from the electrodes. In FIGS. 7A-7C, the techniques of this disclosure will be described as performed by processing circuitry 50 of IMD 10, however, in some examples these techniques may also be performed in whole or in part by one or more of the processing circuitry of another device of the medical system (e.g., an external device, cloud computing device, etc.) Processing circuitry of IMD 10 may be configured to determine, based at least in part on the one or more sensor signals, a waveform corresponding to a breathing pattern of the patient. For example, IMD 10 may be configured to determine impedance waveform 700 corresponding to a breathing pattern of the patient based at least in part on the tissue impedance signals.

Impedance waveform 700 may consist of a plurality of tissue impedance values 710A-N (all together, tissue impedance values 710) from the one or more tissue impedance signals. Tissue impedance values 710 in impedance waveform 700 may be collected over a time period, for example two minutes. A medical system may continuously analyze impedance waveform 700. For example, tissue impedance values 710 may be stored in a buffer of tissue impedance values, where the buffer stores tissue impedance values 710 measured over a most recent time period (e.g., two minutes) for analysis by the medical system to form impedance waveform 700. The buffer may continuously update with new tissue impedance values 710, discarding older tissue impedance values 710, as tissue impedance measurements are made. In some examples, the buffer may update based on identifying tissue impedance measurements that are most clinically relevant.

FIG. 7B is a graph illustrating example signals derived from the example impedance waveform of FIG. 7A, in accordance with one or more techniques of this disclosure. Processing circuitry of IMD 10 may be configured to determine, based on the impedance waveform, an envelope signal 730. For example, processing circuitry 50 may subtract a moving average from the impedance waveform to subtract out low-frequency trends. Processing circuitry 50 may then take an absolute value of the resulting signal to generate an absolute value signal 720. Processing circuitry 50 may then determine envelope signal 730 based on absolute value signal 720.

In some examples, processing circuitry 50 may apply a low-pass filter to absolute value signal 720 to generate envelope signal 730. In some examples, processing circuitry 50 may apply a moving average filter or sample and hold followed by filtering to absolute value signal 720 or adaptive demodulation using the respiration rate sinusoid to generate envelope signal 730. In general, envelope signal 730 may oscillate to some degree around a non-zero value. For example, envelope signal 730 may oscillate around median 732. Processing circuitry 50 may calculate median 732 as an average of envelope signal 730.

Although attributed to processing circuitry 50 of IMD 10, in some examples another computing device in communication with IMD 10 may be configured to perform one or more of the techniques described herein. For example, IMD 10 may send a waveform representing a breathing pattern of patient 4 (breathing waveform) to one or more servers and/or cloud computing devices for data processing. The one or more cloud computing devices may determine envelope signal 730 using the one or more techniques described above. In some examples, the one or more cloud computing devices may determine envelope signal 730 using one or more deep learning regression networks, where the one or more cloud computing devices apply the breathing waveform the deep learning networks as input, and determine as output from the deep learning networks, envelope signal 730. In some examples, battery power of IMD 10 may be conserved by sending data to an external device for processing.

The processing circuitry may also be configured to determine a SDB index based at least in part on the envelope signal. The SDB index may be a measure of the quality or abnormality of patient 4's breathing. For example, the SDB index may be a number (e.g., between 1 and 10, between 1 and 100), where the smaller the number is the more normal patient 4's breathing is, and the higher the number, the more abnormal the breathing is. In some examples, the SDB index may be a classification of patient 4's breathing. For example, the SDB index may include terms referencing the type of breathing detected (e.g., “deep breathing”, “shallow breathing”, “normal breathing”, “high likelihood of apnea”, “Cheyne-Stokes breathing”) or numbers correlated to those terms or breathing types.

In order to determine a SDB index based on envelope signal 730, processing circuitry 50 may calculate one or more thresholds. For example, processing circuitry 50 may calculate upper threshold 734A and lower threshold 734B (together thresholds 734). In some examples, thresholds 734 may be calculated as a percentage above and below median 732. In some examples, upper threshold 734A is ten percent above median 732 and lower threshold 734B is ten percent below median 732.

Processing circuitry may be configured to detect the one or more SDB episodes based on a difference between envelope signal 730 and median 732. For example, processing circuitry 50 may calculate a set of difference values between each data point of envelope signal 730 and median 732, and sum the set of difference values. That is, the difference may include the set of difference values for the duration of the current envelope signal 730. In some examples, the time period may consist of a portion of the duration of envelope signal 730. In response to the sum exceeding a threshold, processing circuitry 50 may determine that the patient experienced an SDB episode.

In some examples, processing circuitry 50 may calculate each difference value of the set of difference values by subtracting the median value multiplied by a percentage from each value of the set of values in envelope signal 730 over the duration of envelope signal 730. For example, processing circuitry 50 may determine which values of the envelope signal are outside of the threshold ranges, that is, above threshold 734A or below threshold 734B. Processing circuitry 50 may determine a cumulative sum of the differences between envelope signal 730 and thresholds 734. The cumulative sum may reflect a cumulative sum of the one or more areas 736 between the curve of envelope signal 730 and thresholds 734. For example, in the example of FIG. 7B, the cumulative sum may be a sum of the five areas 736 shown. If a patient is breathing normally, areas 736 will be low or close to zero. Processing circuitry 50 may determine the SDB index for the patient based on the cumulative sum of areas 736. For example, the higher the cumulative sum of areas 736, the higher the SDB index may be. In some examples, a database in memory of IMD 10 contains reference charts, spreadsheets, or other predetermined data for correlating cumulative sums to SDB indexes. Processing circuitry 50 may compare a measured cumulative sum of areas 736 to values in a table in memory to determine a corresponding SDB index.

In some examples, processing circuitry 50 may determine a time duration that envelope signal 730 is above or below thresholds 734. For example, processing circuitry 50 may identify one or more time periods 740 in envelope signal 730 where envelope signal 730 is above or below thresholds 734. Processing circuitry 50 may sum all time periods 740 in envelope signal 730 to calculate the time duration that envelope signal 730 is outside of the threshold range. Processing circuitry 50 may determine the SDB index for the patient based on the sum of time periods 740. For example, the longer the time duration that envelope signal 730 is outside the threshold range, the higher the SDB index may be. In some examples, a database in memory of IMD 10 contains reference charts, spreadsheets, or other predetermined data for correlating time durations outside the threshold range to SDB indexes. Processing circuitry 50 may compare a measured sum of time periods 740 to values in a table in memory to determine a corresponding SDB index.

In some examples, processing circuitry is configured to detect one or more SDB episodes based on the envelope signal. In some examples, processing circuitry 50 may detect an SDB episode based on the sum of the set of difference values exceeding a threshold. For example, if the cumulative sum of areas 736 exceeds a threshold value, processing circuitry 50 may determine that an SDB episode has occurred. In some examples, processing circuitry 50 may detect an SDB episode based on determining that a time period in which envelope signal 730 exceeds thresholds 734 exceeds a threshold amount of time. For example, if the sum of time periods 740 exceeds a threshold amount of time, processing circuitry 50 may determine that an SDB episode has occurred. Processing circuitry 50 may continuously analyze impedance waveforms to detect one or more SDB episodes.

Processing circuitry 50 may determine a quantification of the one or SDB episodes. For example, processing circuitry 50 may save a count in memory of the number of SDB episodes detected within a given time frame, e.g., one week. In some examples, processing circuitry 50 may store a duration in memory in which SDB episodes or an SDB episode persisted. For example, processing circuitry 50 may determine that the patient experienced an SDB episode for five minutes during a single night. In some examples, processing circuitry 50 may determine that the patient experienced at least one SDB episode every night for a week.

Processing circuitry may be configured to determine the SDB index based on the quantification of the one or more SDB episodes. For example, the higher the number of SDB episodes or the longer the patient experienced SDB episodes, the higher the SDB index. In examples where the SDB index is a classification, processing circuitry 50 may determine the classification based on the quantification of SDB episodes exceeding certain thresholds. For example, in response to determining that the patient experienced SDB episodes every night for a week, processing circuitry 50 may determine that the SDB index is “high likelihood of sleep apnea.” In some examples, in response to determining that the duration that the patient experienced SDB episodes in a week was twenty seconds total, processing circuitry 50 may determine that the SDB index is “low likelihood of sleep apnea.”

In some examples, processing circuitry may be configured to activate a sleep study mode of IMD 10 in response to determining that the quantification of the SDB episodes exceeds a threshold. For example, processing circuitry 50 may activate a sleep study mode of IMD 10 in response to determining that the patient experienced fifteen SDB episodes in the past week. In some examples, processing circuitry 50 may activate a sleep study mode of IMD 10 in response to determining that the patient experienced SDB episodes for fifty minutes in the past week. In some examples, processing circuitry 50 may activate a sleep study mode in response to user input, e.g., via an external device in communication with IMD 10.

During normal operation, IMD 10 may collect only minimal data from patient tissue, or only minimal physiological parameters from the patient using sensors of IMD 10 in order to conserve battery power. IMD 10 may also only operate intermittently, e.g., 20 minutes at a time once a day. In some examples, a user of external device 12 may adjust settings to IMD 10 to control a tradeoff between data collection and battery longevity. For example, a user may adjust the settings of IMD 10 to change the type of data collected by IMD 10 (e.g., when to start or stop impedance measurements, accelerometer measurements, ECG measurements, or measurements of any other sensors of IMD 10), to change a length of time data is collected by IMD 10 (e.g., impedance measurements may only be collected for 30 minute increments or only a certain number of times per week, etc.), and/or to change the amount of data processing performed by processors 50 of IMD 10 (e.g., a breathing waveform may be sent to an external device for signal processing).

When the sleep study mode is activated, IMD 10 may activate multiple sensors and/or sensor types and collect signals indicative of multiple physiological parameters. For example, in the sleep study mode, IMD 10 may activate sensing of at least a first physiological parameter other than respiration (e.g., heart rate). In some examples, in the sleep study mode, IMD 10 may increase a resolution of sensing of second physiological parameter other than respiration. In some examples, IMD 10 may activate these and other sensors for an extended period, e.g., during nighttime when the patient is asleep. Various methods may be used to determine if the patient is asleep, e.g., breathing rate, heart rate, body temperature, body motion, etc. In some examples, other physiological parameters include one or more of a heart rate of the patient, an activity of the patient, a posture of the patient, a blood pressure of the patient, a body fat percentage of the patient, a tissue impedance of the patient, an electrocardiogram of the patient, or an intracardiac electrogram of the patient.

IMD 10 may be able to more accurately determine an SDB index for the patient when measuring multiple physiological parameters. The processing circuitry may be configured to determine the SDB index based on the quantification of the one or more SDB episodes, as well as one or more other physiological parameter measurements. For example, in a sleep study mode, processing circuitry 50 may measure impedance waveforms indicative of patient breathing patterns, as well as a heart rate of the patient, blood oxygen saturation (SpO2) of the patient, and a blood pressure of the patient. In some examples, a heart rate threshold table may be preloaded in memory of IMD 10 that correlates heart rates or heart rate patterns to SDB indexes. For example, a fast, irregular heartbeat during a sleep study may be correlated with a higher SDB index, or a higher likelihood that the patient may experience a HF event. In some examples, a large cyclical variation of heart rate may indicate compensation during a SDB episode. Similar tables may be preloaded into memory of IMD 10 for other physiological parameters for IMD to use in determining an SDB index based on the physiological parameters. In this way, the IMD may continuously monitor SDB indexes for a long period, e.g., several years. The IMD may monitor SDB nightly, and may initiate a sleep study mode whenever necessary.

In some examples, the processing circuitry may be configured to determine the SDB index by applying a machine learning model to the quantification of the one or more SDB episodes and at least one other physiological parameter. For example, a quantification of the one or more SDB episodes may be the number fifteen, representing fifteen hours in the past week in which the patient experienced SDB episodes, as determined by IMD 10. IMD 10 may measure a resting heart rate of ninety beats per minute. Processing circuitry 50 may apply the quantification of the SDB episodes and the heart rate of the patient as input to the machine learning model and determine, as output, the SDB index of the patient.

After performing the sleep study, IMD may save data collected during the sleep study to a databased in memory accessible by a physician/care provider for review. In this way, a physician may be able to recommend therapy to a patient faster than if the patient had to schedule an in person sleep study. For example, a physician may determine whether to prescribe a CPAP therapy or other therapy. A physician may also check for compliance of CPAP therapy. In some examples, the IMD may provide a nightly trend of the SDB index, which a physician may correlate with the patient's AF burden as well as the occurrence of other arrhythmias. In some examples, a physician may be able to measure the effectiveness of a CPAP therapy by analyzing the data from the IMD both before and after CPAP therapy is administered. Earlier prescription of therapy may reduce instances of HF, hospitalization, and death.

Processing circuitry of the medical system may determine a heart condition status of patient 4 based at least in part on the SDB index. In some examples, the heart condition status may represent a risk of HF. For example, the heart condition status may be a number (e.g., between 1 and 100), representing a percentage risk that patient 4 will experience HF. In some examples, the heart condition status may be a string classification (e.g., “at risk of HF”, “not at risk of HF”, “high risk of HF event”) or numbers correlated to those classifications in memory.

In some examples, processing circuitry 50 may determine a heart condition status algorithmically via one or more lookup tables in memory. For example, a database in memory may include one or more tables correlating SDB indexes to heart condition statuses. In some examples, a database in memory may include one or more tables correlating other physiological parameter measurements to heart condition statuses. Processing circuitry 50 may cross-reference SDB indexes and/or other physiological parameter values in the tables in memory to determine a heart condition status.

In some examples, processing circuitry 50 may be configured to determine the heart condition status of the patient based on application of one or more machine learning models to the SDB index and one or more other physiological parameters of the patient. For example, processing circuitry 50 may apply the SDB index and the one or more other physiological parameters of the patient as input to the machine learning model. Processing circuitry 50 may determine, as output from the machine learning model, the heart condition status of the patient.

Processing circuitry 50 may be configured to determine if the heart condition status exceeds a threshold. For example, in examples where the heart condition status is represented by a number (e.g., one through one hundred as a percent risk of HF), processing circuitry 50 may determine if the heart condition status exceeds a value of sixty. In response to determining that the heart condition status exceeds a threshold, processing circuitry 50 may be configured to save one or more segments of the impedance waveform from which the heart condition status was determined to the memory. In examples where the heart condition status is a classification category (e.g., “high risk of HF”), processing circuitry 50 may be configured to save the one or more segments of the impedance waveform in response to the heart condition status falling into one or more particular classification categories. A physician or user of an external device may be able to access the memory and the saved impedance waveform for further analysis or diagnosis.

The processing circuitry 50 may also be configured to generate an alert (e.g., a notification, a status indicator, an alarm, etc.). For example, processing circuitry 50 may generate an alert in response to one or more of the SDB index exceeding a threshold, detection of an SDB episode in the patient, and/or a heart condition status exceeding a threshold. In some examples, where the SDB index and/or the heart condition status are represented by a classification category (e.g., “high likelihood of apnea”, “high risk of HF event”), processing circuitry 50 may be configured to generate an alert in response to the SDB index and/or the heart condition status falling into one or more classifications. In a non-limiting example, the alert may include text or graphics information that communicates the impedance waveform, SDB index, SDB episode, heart condition status, or other status of the patient. In some examples, IMD 10 may transmit the alert to another computing device (e.g. the external device). In some examples where a computing device other than IMD 10 determines the SDB index, SDB episode, and/or heart condition status, that computing device may simply generate the alert or may further transmit the alert to another device of the medical system. In some examples, a computing device may transmit an alert to IMD 10 that instructs IMD 10 to take some action in response to the alert. The alert may indicate one or more breathing or heart condition statuses. For example, an alert may trigger for high risk of HF and detection of an SDB episode, and/or a different alert may trigger indicating that the patient had a SDB index of “high likelihood of apnea.” In some examples, the type of alert may correspond to the type of status it communicates. For example, an alert for a “high risk of HF” may include a high pitched sound, an alert for a “medium risk of HF” may include a medium-pitched sound, and an alert for a “low risk of HF” may include a soft, low-pitched sound or no sound at all. The alerts may be communicated directly to the patient or to the clinician through a variety of methods including notifications, audible tones, handheld devices and automatic or on-demand telemetry to computerized communication network. In some examples, the alert may include an alarm, such as an audible alarm or visual alarm.

FIG. 7C is a graph illustrating an example phase plot 750 derived from the example envelope signal of FIG. 7B, in accordance with one or more techniques of this disclosure. In some examples, processing circuitry 50 may determine phase plot 750 of the envelope signal over a time period.

For example, processing circuitry 50 may identify a pair of points separated by some number N samples. That pair of points may be one point on phase plot 750. Processing circuitry 50 may repeat this for every sample of the waveform and plot {Env(x), Env(x+N)}. Processing circuitry 50 may perform this operation for multiple values of N, which may tune phase plot 750 for the cycle of the envelope. A cycle may appear close to a circle when N is ¼th of the cycle length of the envelope. A lack of a cycle may appear as one point along the 45 degree line. The amplitude of the envelope may determine the diameter of the circle for a cyclical envelope.

Processing circuitry 50 may determine if phase plot 750 shows periodic trends over the time period encompassing the duration of the current envelope signal. In response to determining that phase plot 750 shows periodic trends, processing circuitry 50 may detect an SDB episode. The periodic trends may appear as a cycle in phase plot 750. In some examples, processing circuitry 50 may determine an SDB index by determining a diameter of a circle in phase plot 750. In some examples, processing circuitry 50 may determine an SDB index by determining an area of the best fit circle of phase plot 750.

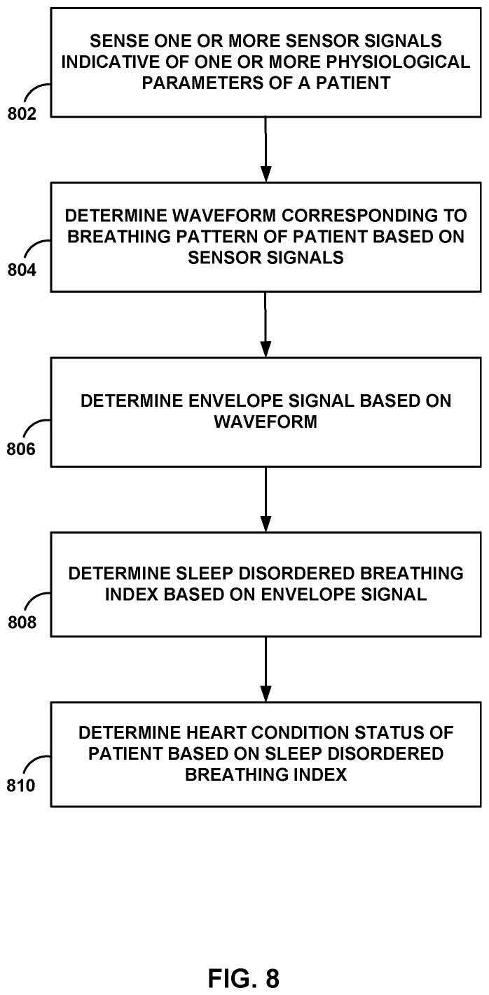

FIG. 8 is a flow diagram illustrating an example operation for determining a heart condition status of a patient based on a subcutaneous tissue impedance signal, in accordance with one or more techniques of this disclosure. Although described as being performed by IMD 10, one or more of the various example techniques described with reference to FIG. 6 may be performed by any one or more of IMD 10, external device 12, or server 94, e.g., by the processing circuitry of any one or more of these devices.

In some examples, a method includes sensing, by one or more sensors of a medical device, one or more sensor signals indicative of one or more physiological parameters of a patient (802). For example, a medical device may receive one or more subcutaneous tissue impedance signals over time corresponding to a breathing pattern of a patient. In some examples, a medical system may include an IMD. e.g., IMD 10 of FIG. 1. IMD 10 may include one or more electrodes on its housing, or may be coupled to one or more leads that carry one or more electrodes for sensing one or more subcutaneous tissue impedance signals over time. IMD 10 may be configured for subcutaneous implantation outside of a thoracic cavity of the patient. IMD 10 may receive the one or more signals indicative of subcutaneous tissue impedance from the electrodes.

The method may include determining, by processing circuitry of the medical device, based at least in part on the one or more sensor signals, a waveform corresponding to a breathing pattern of the patient (804). For example, processing circuitry of the IMD may determine an impedance waveform based at least in part on the one or more tissue impedance signals. The impedance waveform may include a plurality of tissue impedance values from the subcutaneous impedance signals accumulated over a period of time. For example, the impedance values may be stored in a buffer of impedance values, where the buffer stores impedance values measured over a most recent time period for analysis to form the impedance waveform. In some examples, the impedance waveform includes the last ten seconds, two minutes, or any other time period of impedance values.

The method may further include determining an envelope signal based on the waveform (806). For example, IMD 10 may subtract a moving average from the impedance waveform to subtract out low-frequency trends. IMD 10 may then take an absolute value of the resulting signal to generate an absolute value signal. Finally, IMD 10 may determine the envelope signal based on the absolute value signal. In some examples, the method includes applying a low-pass filter to the absolute value signal to generate the envelope signal. In some examples, the method includes applying a moving average filter to the absolute value signal to generate the envelope signal. In general, the envelope signal may oscillate to some degree around a non-zero value. For example, the envelope signal may oscillate around an average or median value. The method may include calculating a median value as an average of the envelope signal.

The method may further include determining an SDB index based at least in part on the envelope signal (808). The SDB index may be a measure of the quality or abnormality of the patient's breathing. For example, the SDB index may be a number (e.g., between 1 and 10, between 1 and 100), where the smaller the number is the more normal the patient's breathing is, and the higher the number, the more abnormal the breathing is. In some examples, the SDB index may be a classification of the patient's breathing. For example, the SDB index may include terms referencing the type of breathing detected (e.g., “deep breathing”, “shallow breathing”, “normal breathing”, “high likelihood of apnea”, “Cheyne-Stokes breathing”) or numbers correlated to those terms or breathing types.

In order to determine a SDB index based on the envelope signal, the method may include calculating one or more median thresholds. For example, IMD 10 may calculate an upper threshold and a lower threshold. In some examples, the median thresholds may be calculated as a percentage above and below the median of the envelope signal. In some examples, the upper threshold is ten percent above the median and the lower threshold is ten percent below the median.

The method may also include detecting one or more SDB episodes based on a difference between the envelope signal and the median. For example, the method may include calculating a set of difference values between each data point of the envelope signal and the median, and summing the set of difference values. That is, the difference may include the set of difference values for the duration of the current the envelope signal. In some examples, the time period may consist of a portion of the duration of the envelope signal. In response to the sum exceeding a threshold, the method may include determining that the patient experienced an SDB episode.

In some examples, the method may include calculating each difference value of the set of difference values by subtracting the median value multiplied by a percentage from each value of the set of values in the envelope signal over the duration of the envelope signal. For example, the method may include determining which values of the envelope signal are outside of the median threshold range, that is, above the upper threshold or below the lower threshold. IMD 10 may determine a cumulative sum of the differences between the envelope signal and the median thresholds. The cumulative sum may reflect a cumulative sum of the one or more the areas between the curve of the envelope signal and the median thresholds. For example, in the example of FIG. 7B, the cumulative sum may be a sum of the five the areas shown. If a patient is breathing normally, the areas will be low or close to zero. The method may include determining the SDB index for the patient based on the cumulative sum of the areas. For example, the higher the cumulative sum of the areas, the higher the SDB index may be. In some examples, a database in memory of IMD 10 contains reference charts, spreadsheets, or other predetermined data for correlating cumulative sums to SDB indexes. The method may include comparing a measured cumulative sum of the areas to values in a table in memory to determine a corresponding SDB index.

In some examples, the method may include determining a time duration that the envelope signal is above or below the median thresholds. For example, IMD 10 may identify one or more the time periods in the envelope signal where the envelope signal is above or below the median thresholds. IMD 10 may sum all the time periods in the envelope signal to calculate the time duration that the envelope signal is outside of the threshold range. IMD 10 may determine that the sum of the time periods exceeds a threshold amount of time. The threshold amount of time may be saved to a database in memory. IMD 10 may determine the SDB index for the patient based on the sum of the time periods. For example, the longer the time duration that the envelope signal is outside the threshold range, the higher the SDB index may be. In some examples, a database in memory of IMD 10 contains reference charts, spreadsheets, or other predetermined data for correlating time durations outside the threshold range to SDB indexes. IMD 10 may compare a measured sum of the time periods to values in a table in memory to determine a corresponding SDB index.

In some examples, the method includes detecting one or more SDB episodes based on the envelope signal. In some examples, the method includes detecting an SDB episode based on the sum of the set of difference values exceeding a threshold. For example, if the cumulative sum of the areas exceeds a threshold value, IMD 10 may determine that an SDB episode has occurred. In some examples, the method includes detecting an SDB episode based on determining that a time period in which the envelope signal exceeds the median thresholds exceeds a threshold amount of time. For example, if the sum of the time periods exceeds a threshold amount of time, IMD 10 may determine that an SDB episode has occurred. IMD 10 may continuously analyze waveforms corresponding to breathing patterns of the patient to detect one or more SDB episodes.

The method may include determining a quantification of the one or SDB episodes. For example, IMD 10 may save a count in memory of the number of SDB episodes detected within a given time frame, e.g., one week. In some examples, IMD 10 may store a duration in memory in which SDB episodes or an SDB episode persisted. For example, IMD 10 may determine that the patient experienced an SDB episode for five minutes during a single night. In some examples, IMD 10 may determine that the patient experienced at least one SDB episode every night for a week.

The method may also include determining the SDB index based on the quantification of the one or more SDB episodes. For example, the higher the number of SDB episodes or the longer the patient experienced SDB episodes, the higher the SDB index. In examples where the SDB index is a classification, IMD 10 may determine the classification based on the quantification of SDB episodes exceeding certain thresholds. For example, in response to determining that the patient experienced SDB episodes every night for a week, IMD 10 may determine that the SDB index is “high likelihood of sleep apnea.” In some examples, in response to determining that the duration that the patient experienced SDB episodes in a week was twenty seconds total, IMD 10 may determine that the SDB index is “minor sleep apnea.”

In some examples, the method may include determining the SDB index by applying a machine learning model to the quantification of the one or more SDB episodes and at least one other physiological parameter. For example, a quantification of the one or more SDB episodes may be the number fifteen, representing fifteen hours in the past week in which the patient experienced SDB episodes, as determined by IMD 10. IMD 10 may measure a resting heart rate of ninety beats per minute. In some examples, IMD 10 may measure a number or duration of cyclical fluctuations of the heart rate. IMD 10 may apply the quantification of the SDB episodes and the heart rate of the patient as input to the machine learning model and determine, as output, the SDB index of the patient.

The method may also include determining a heart condition status of the patient based at least in part on the SDB index (810). In some examples, the heart condition status may represent a risk of HF. For example, the heart condition status may be a number (e.g., between 1 and 100), representing a percentage risk that patient 4 will experience HF. In some examples, the heart condition status may be a string classification (e.g., “at risk of HF”, “not at risk of HF”, “high risk of HF event”) or numbers correlated to those classifications in memory.

In some examples, the method may include determining a heart condition status algorithmically via one or more lookup tables in memory. For example, a database in memory may include one or more tables correlating SDB indexes to heart condition statuses. In some examples, a database in memory may include one or more tables correlating other physiological parameter measurements to heart condition statuses. IMD 10 may cross-reference SDB indexes and/or other physiological parameter values in the tables in memory to determine a heart condition status.

In some examples, the method may include determining the heart condition status of the patient based on application of one or more machine learning models to the SDB index and one or more other physiological parameters of the patient. For example, IMD 10 may apply the SDB index and the one or more other physiological parameters of the patient as input to the machine learning model. IMD 10 may determine, as output from the machine learning model, the heart condition status of the patient.

The method may include determining if the heart condition status exceeds a threshold. For example, in examples where the heart condition status is represented by a number (e.g., one through one hundred as a percent risk of HF), IMD 10 may determine if the heart condition status exceeds a value of sixty. In response to determining that the heart condition status exceeds a threshold, IMD 10 may be configured to save one or more segments of the impedance waveform from which the heart condition status was determined to the memory. In examples where the heart condition status is a classification category (e.g., “high risk of HF”), IMD 10 may be configured to save the one or more segments of the impedance waveform in response to the heart condition status falling into one or more particular classification categories. A physician or user of an external device may be able to access the memory and the saved impedance waveform for further analysis or diagnosis.

The method may also include generating an alert (e.g., a notification, a status indicator, an alarm, etc.). For example, IMD 10 may generate an alert in response to one or more of the SDB index exceeding a threshold, detection of an SDB episode in the patient, and/or a heart condition status exceeding a threshold. In some examples, where the SDB index and/or the heart condition status are represented by a classification category (e.g., “high likelihood of apnea”, “high risk of HF event”), IMD 10 may be configured to generate an alert in response to the SDB index and/or the heart condition status falling into one or more classifications. In a non-limiting example, the alert may include text or graphics information that communicates the impedance waveform, SDB index, SDB episode, heart condition status, or other status of the patient. In some examples, IMD 10 may transmit the alert to another computing device (e.g. the external device). In some examples where a computing device other than IMD 10 determines the SDB index, SDB episode, and/or heart condition status, that computing device may simply generate the alert or may further transmit the alert to another device of the medical system. In some examples, a computing device may transmit an alert to IMD 10 that instructs IMD 10 to take some action in response to the alert. The alert may indicate one or more breathing or heart condition statuses. For example, an alert may trigger for high risk of HF and detection of an SDB episode, and/or a different alert may trigger indicating that the patient had a SDB index of “high likelihood of apnea.” In some examples, the type of alert may correspond to the type of status it communicates. For example, an alert for a “high risk of HF” may include a high pitched sound, an alert for a “medium risk of HF” may include a medium-pitched sound, and an alert for a “low risk of HF” may include a soft, low-pitched sound or no sound at all. The alerts may be communicated directly to the patient or to the clinician through a variety of methods including notifications, audible tones, handheld devices and automatic or on-demand telemetry to computerized communication network. In some examples, the alert may include an alarm, such as an audible alarm or visual alarm.

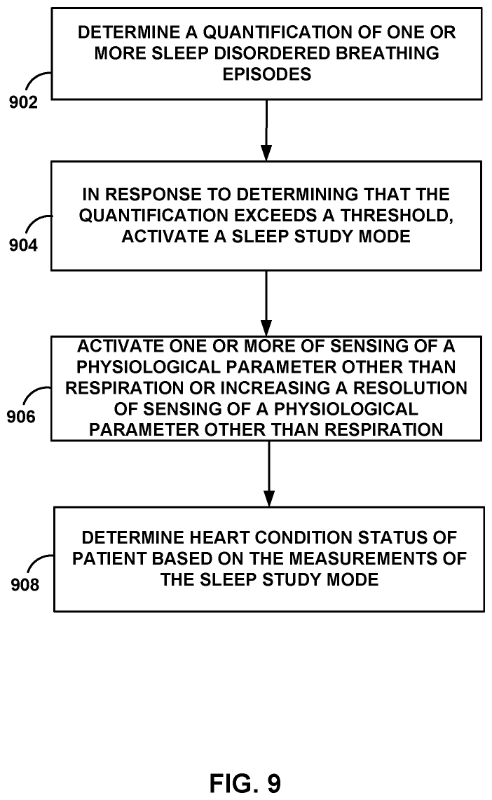

FIG. 9 is a flow diagram illustrating an example operation for initiating a sleep study mode in an IMD, in accordance with one or more techniques of this disclosure. In some examples the method may include determining a quantification of one or more SDB episodes, as described above (902). In response to determining that the quantification exceeds a threshold, the method may include activating a sleep study mode (904). For example, IMD 10 may activate a sleep study mode of IMD 10 in response to determining that the patient experienced fifteen SDB episodes in the past week. In some examples, IMD 10 may activate a sleep study mode of IMD 10 in response to determining that the patient experienced SDB episodes for fifty minutes in the past week. In some examples, IMD 10 may activate a sleep study mode in response to user input, e.g., via an external device in communication with IMD 10.

During normal operation, IMD 10 may collect only minimal data from patient tissue, or only minimal physiological parameters from the patient using sensors of IMD 10 in order to conserve battery power. IMD 10 may also only operate intermittently, e.g., 20 minutes at a time once a day. When the sleep study mode is activated, IMD 10 may activate multiple sensors and/or sensor types and collect signals indicative of multiple physiological parameters.

For example, the method may include activating, in the sleep study mode, sensing of at least a first physiological parameter other than respiration (e.g., heart rate) (906). In some examples, the method includes increasing, in the sleep study mode, a resolution of sensing of second physiological parameter other than respiration. In some examples, IMD 10 may activate these and other sensors for an extended period, e.g., during nighttime when the patient is asleep. Various methods may be used to determine if the patient is asleep, e.g., breathing rate, heart rate, body temperature, body motion, etc. In some examples, other physiological parameters include one or more of a heart rate of the patient, an activity of the patient, a posture of the patient, a blood pressure of the patient, a body fat percentage of the patient, a tissue impedance of the patient, an electrocardiogram of the patient, or an intracardiac electrogram of the patient.

IMD 10 may be able to more accurately determine an SDB index for the patient when measuring multiple physiological parameters. The method may include determining the SDB index based on the quantification of the one or more SDB episodes, as well as one or more other physiological parameter measurements. For example, in a sleep study mode, IMD 10 may measure impedance waveforms indicative of patient breathing patterns, as well as a heart rate patterns of the patient, blood oxygen saturation (SpO2) of the patient, and a blood pressure of the patient. In some examples, a heart rate threshold table may be preloaded in memory of IMD 10 that correlates heart rates or heart rate patterns to SDB indexes. For example, a fast, irregular heartbeat during a sleep study may be correlated with a higher SDB index, or a higher likelihood that the patient may experience a HF event. In some examples, a large cyclical variation of heart rate may indicate compensation during a SDB episode. Similar tables may be preloaded into memory of IMD 10 for other physiological parameters for IMD to use in determining an SDB index based on the physiological parameters. In this way, the IMD may continuously monitor SDB indexes for a long period, e.g., several years. The IMD may monitor SDB nightly, and may initiate a sleep study mode whenever necessary.

The method may further include determining a heart condition status of the patient based on the measurements of the sleep study mode (908). For example, in a sleep study mode, IMD 10 may determine an SDB index of the patient, as well as measure a heart rate of the patient, and a blood pressure of the patient. In some examples, IMD 10 may also collect an ECG of the patient. In some examples, an SDB index table may be preloaded in memory of IMD 10 that correlates SDB indexes to heart condition statuses. Similar tables may be preloaded into memory of IMD 10 for other physiological parameters for IMD to use in determining an SDB index based on the physiological parameters. IMD 10 may cross-reference the measurements of the sleep study mode with their corresponding tables in memory to determine a heart condition status. In some examples, the method includes determining a heart condition status of the patient based on applying the measurements of the sleep study mode to a machine learning algorithm as input. The machine learning algorithm may output the heart condition status of the patient.

After performing the sleep study, IMD may save data collected during the sleep study to a databased in memory accessible by a physician/care provider for review. In this way, a physician may be able to recommend therapy to a patient faster than if the patient had to schedule an in person sleep study. For example, a physician may determine whether to prescribe a CPAP therapy or other therapy. Earlier prescription of therapy may reduce instances of HF, hospitalization, and death.

Various examples have been described. However, one skilled in the art will appreciate that various modifications may be made to the described examples without departing from the scope of the claims. For example, although described primarily with reference to subcutaneous impedance (as a measure of a breathing pattern), in some examples other physiological parameters may be considered with subcutaneous impedance to detect worsening heart failure. Examples of other physiological parameters and techniques for detecting worsening heart failure based on these parameters in combination with impedance are described in commonly-assigned U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, both of which are incorporated herein by reference in their entirety.

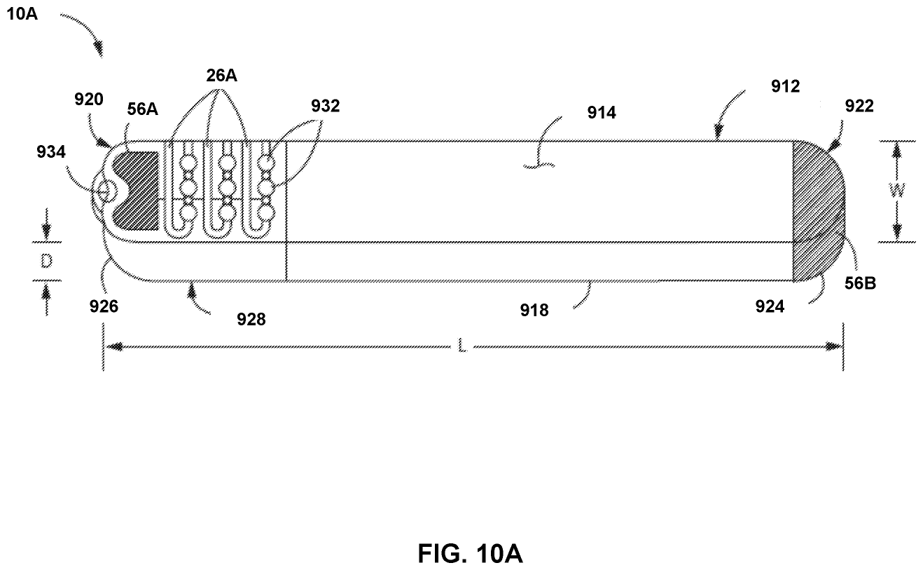

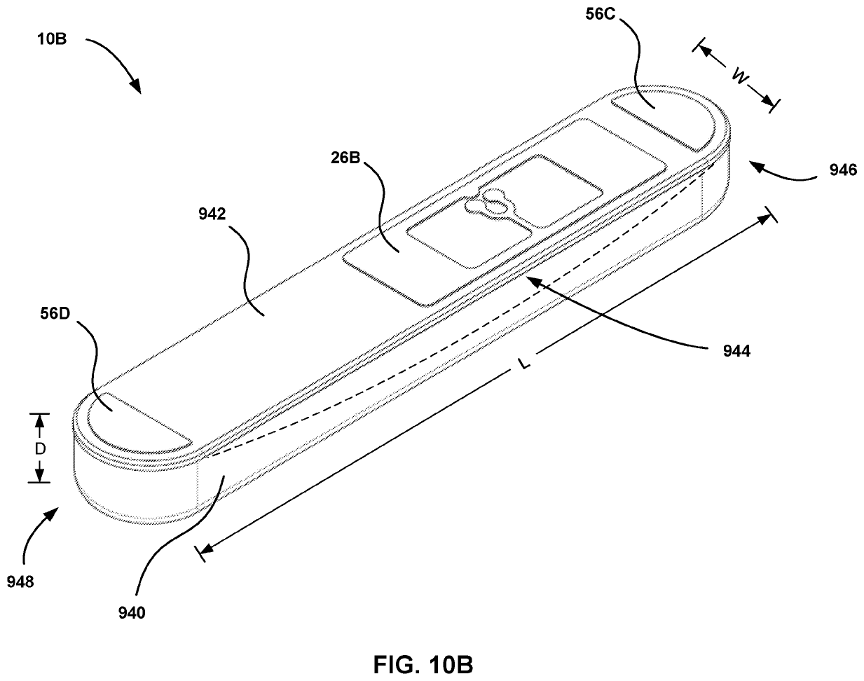

FIG. 10A is a perspective drawing illustrating an IMD 10A, which may be an example configuration of IMD 10 of FIGS. 1-3 as an ICM. In the example shown in FIG. 10A, IMD 10A may be embodied as a monitoring device having housing 912, proximal electrode 16A and distal electrode 16B. Housing 912 may further comprise first major surface 914, second major surface 918, proximal end 920, and distal end 922. Housing 912 encloses electronic circuitry located inside the IMD 10A and protects the circuitry contained therein from body fluids. Housing 912 may be hermetically sealed and configured for subcutaneous implantation. Electrical feedthroughs provide electrical connection of electrodes 16A and 16B.

In the example shown in FIG. 10A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 10A includes radial asymmetries (notably, the rectangular shape) along the longitudinal axis that maintains the device in the proper orientation following insertion. For example, the spacing between proximal electrode 16A and distal electrode 16B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 914 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for case of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 10A, once inserted within the patient, the first major surface 914 faces outward, toward the skin of the patient while the second major surface 918 is located opposite the first major surface 914. In addition, in the example shown in FIG. 10A, proximal end 920 and distal end 922 are rounded to reduce discomfort and irritation to surrounding tissue once inserted under the skin of the patient. IMD 10A, including instrument and method for inserting IMD 10A is described, for example, in U.S. Patent Publication No. 2014/0276928, incorporated herein by reference in its entirety.

Proximal electrode 16A is at or proximate to proximal end 920, and distal electrode 16B is at or proximate to distal end 922. Proximal electrode 16A and distal electrode 16B are used to sense cardiac EGM signals, e.g., ECG signals, and measure interstitial impedance thoracically outside the ribcage, which may be sub-muscularly or subcutaneously. EGM signals and impedance measurements may be stored in a memory of IMD 110A, and data may be transmitted via integrated antenna 26A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, from any implanted location. Housing 912 may house the circuitry of IMD 10 illustrated in FIG. 3.

In the example shown in FIG. 10A, proximal electrode 16A is at or in close proximity to the proximal end 920 and distal electrode 16B is at or in close proximity to distal end 922. In this example, distal electrode 16B is not limited to a flattened, outward facing surface, but may extend from first major surface 914 around rounded edges 924 and/or end surface 926 and onto the second major surface 918 so that the electrode 56B has a three-dimensional curved configuration. In some examples, electrode 16B is an uninsulated portion of a metallic, e.g., titanium, part of housing 912.

In the example shown in FIG. 10A, proximal electrode 16A is located on first major surface 914 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 914 similar to that shown with respect to proximal electrode 16A.

The various electrode configurations allow for configurations in which proximal electrode 16A and distal electrode 16B are located on both first major surface 914 and second major surface 918. In other configurations, such as that shown in FIG. 10A, only one of proximal electrode 16A and distal electrode 16B is located on both major surfaces 914 and 918, and in still other configurations both proximal electrode 16A and distal electrode 16B are located on one of the first major surface 914 or the second major surface 918 (e.g., proximal electrode 16A located on first major surface 914 while distal electrode 16B is located on second major surface 918). In another example, IMD 10A may include electrodes on both major surface 914 and 918 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 10A, proximal end 920 includes a header assembly 928 that includes one or more of proximal electrode 16A, integrated antenna 26A, anti-migration projections 932, and/or suture hole 934. Integrated antenna 26A is located on the same major surface (i.e., first major surface 914) as proximal electrode 16A and is also included as part of header assembly 928. Integrated antenna 26A allows IMD 10A to transmit and/or receive data. In other examples, integrated antenna 26A may be formed on the opposite major surface as proximal electrode 16A, or may be incorporated within the housing 912 of IMD 10A. In the example shown in FIG. 10A, anti-migration projections 932 are located adjacent to integrated antenna 26A and protrude away from first major surface 914 to prevent longitudinal movement of the device. In the example shown in FIG. 10A, anti-migration projections 932 include a plurality (e.g., nine) small bumps or protrusions extending away from first major surface 914. As discussed above, in other examples anti-migration projections 932 may be located on the opposite major surface as proximal electrode 16A and/or integrated antenna 26A. In addition, in the example shown in FIG. 10A, header assembly 928 includes suture hole 934, which provides another means of securing IMD 10A to the patient to prevent movement following insertion. In the example shown, suture hole 934 is located adjacent to proximal electrode 16A. In one example, header assembly 928 is a molded header assembly made from a polymeric or plastic material, which may be integrated or separable from the main portion of IMD 10A.

FIG. 10B is a perspective drawing illustrating another IMD 10B, which may be another example configuration of IMD 10 from FIGS. 1-3 as an ICM. IMD 10B of FIG. 10B may be configured substantially similarly to IMD 10A of FIG. 10A, with differences between them discussed herein.

IMD 10B may include a leadless, subcutaneously-implantable monitoring device, e.g. an ICM. IMD 10B includes housing having a base 940 and an insulative cover 942. Proximal electrode 16C and distal electrode 16D may be formed or placed on an outer surface of cover 942. Various circuitries and components of IMD 10B, e.g., described with respect to FIG. 3, may be formed or placed on an inner surface of cover 942, or within base 940. In some examples, a battery or other power source of IMD 10B may be included within base 940. In the illustrated example, antenna 26B is formed or placed on the outer surface of cover 942, but may be formed or placed on the inner surface in some examples. In some examples, insulative cover 942 may be positioned over an open base 940 such that base 940 and cover 942 enclose the circuitries and other components and protect them from fluids such as body fluids. The housing including base 940 and insulative cover 942 may be hermetically sealed and configured for subcutaneous implantation.

Circuitries and components may be formed on the inner side of insulative cover 942, such as by using flip-chip technology. Insulative cover 942 may be flipped onto a base 940. When flipped and placed onto base 940, the components of IMD 10B formed on the inner side of insulative cover 942 may be positioned in a gap 944 defined by base 940. Electrodes 16C and 16D and antenna 26B may be electrically connected to circuitry formed on the inner side of insulative cover 942 through one or more vias (not shown) formed through insulative cover 942. Insulative cover 942 may be formed of sapphire (i.e., corundum), glass, parylene, and/or any other suitable insulating material. Base 940 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 10B, the housing of IMD 10B defines a length L. a width W and thickness or depth D and is in the form of an elongated rectangular prism wherein the length L is much larger than the width W, which in turn is larger than the depth D, similar to IMD 10A of FIG. 10A. For example, the spacing between proximal electrode 16C and distal electrode 16D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 10B, once inserted subcutaneously within the patient, outer surface of cover 942 faces outward, toward the skin of the patient. In addition, as shown in FIG. 10B, proximal end 946 and distal end 948 are rounded to reduce discomfort and irritation to surrounding tissue once inserted under the skin of the patient. In addition, edges of IMD 10B may be rounded.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry (as in QRS complex), as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, ROM, NVRAM, DRAM, SRAM, Flash memory, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Furthermore, although described primarily with reference to examples that provide an impedance score to indicate worsening heart failure in response to detecting impedance changes, other examples may additionally or alternatively automatically modify a therapy in response to detecting worsening heart failure in the patient. The therapy may be, as examples, a substance delivered by an implantable pump, cardiac resynchronization therapy, refractory period stimulation, or cardiac potentiation therapy. These and other examples are within the scope of the following claims.

The following are examples of this disclosure.

Example 1. A system comprising: a medical device comprising one or more sensors configured to sense one or more physiological parameters of a patient; and processing circuitry configured to: sense, by the one or more sensors, one or more sensor signals indicative of the one or more physiological parameters of the patient; determine, based at least in part on the one or more sensor signals, a waveform corresponding to a breathing pattern of the patient; determine, based on the waveform, an envelope signal; determine a sleep disordered breathing index based at least in part on the envelope signal; and determine a heart condition status of the patient based on the sleep disordered breathing index.

Example 2. The system of example 1, wherein the processing circuitry is configured to: detect one or more sleep disordered breathing episodes based on the envelope signal; determine a quantification of one or more sleep disordered breathing episodes; and determine the sleep disordered breathing index based on the quantification.

Example 3. The system of example 2, wherein the processing circuitry is configured to activate a sleep study mode of the medical device in response to determining that the quantification of the sleep disordered breathing episodes exceeds a threshold.

Example 4. The system of example 3, wherein, in the sleep study mode, the medical device is configured to at least one of: activate sensing of a first physiological parameter other than respiration; or increase a resolution of sensing of a second physiological parameter other than respiration.

Example 5. The system of example 4, wherein the processing circuitry is configured to determine the sleep disordered breathing index based on the quantification of the one or more sleep disordered breathing episodes and at least one of the first physiological parameter or the second physiological parameter.

Example 6. The system of example 4, wherein the processing circuitry is configured to determine the sleep disordered breathing index by applying a machine learned model to the quantification of the one or more sleep disordered breathing episodes and at least one of the first physiological parameter or the second physiological parameter.

Example 7. The system of example 2, wherein the processing circuitry is configured to detect the one or more sleep disordered breathing episodes based on a difference between the envelope signal and an average of the envelope signal.

Example 8. The system of example 7, wherein the difference comprises a set of difference values for a time period.

Example 9. The system of example 8, wherein the average of the envelope signal comprises a median of the envelope signal over the time period, and wherein the processing circuitry calculates each difference value of the set of difference values by subtracting the median value multiplied by a percentage from a corresponding value of a set of values in the envelope signal over the time period.

Example 10. The system of any one or more of examples 7-8, wherein the processing circuitry is configured to: determine if a sum of the set of difference values exceeds a threshold; and detect a sleep disordered breathing episode based on the sum of the set of difference values exceeding a threshold.

Example 11. The system of example 2, wherein the processing circuitry is configured to: determine a duration of a time period in which each value of a plurality of values of the envelop signal exceeds a threshold; determine that the time period exceeds a threshold amount of time; and detect a sleep disordered breathing episode of the one or more sleep disordered breathing episodes based on determining that the time period exceeds the threshold amount of time.

Example 12. The system of example 2, wherein the processing circuitry is configured to: determine a phase plot of the envelope signal over the time period; determine if the phase plot shows periodic trends over the time period; and detect a sleep disordered breathing episode of the one or more sleep disordered breathing episodes based on determining that the phase plot shows periodic trends.

Example 13. The system of example 1, further comprising an accelerometer, and wherein the processing circuitry is configured to: collect an accelerometer signal from the accelerometer, wherein the accelerometer signal is indicative of patient movement; determine, based on the accelerometer signal, a time period in which the patient is moving; and determine the sleep disordered breathing index of the patient based on the envelope signal over a portion that does not overlap with the time period.

Example 14. The system of any one or more of examples 1-13, further comprising a memory in communication with the processing circuitry, wherein the processing circuitry is configured to: determine if the sleep disordered breathing index exceeds a threshold; and save, in response to the sleep disordered breathing index exceeding a threshold, the waveform over the time period to the memory.

Example 15. The system of any one or more of examples 1-14, further comprising a memory in communication with the processing circuitry, wherein the processing circuitry is configured to: determine if the sleep disordered breathing index exceeds a threshold; and generate an alert in response to the sleep disordered breathing index exceeding a threshold.

Example 16. The system of any one or more of examples 1 to 15, wherein the processing circuitry is configured to determine the heart condition status of the patient based on application of a machine learned model to the sleep disordered breathing index and one or more other physiological parameters of the patient.

Example 17. The system of example 16, where the physiological parameters include one or more of a heart rate of the patient, an activity of the patient, a posture of the patient, a blood pressure of the patient, a body fat percentage of the patient, an electrocardiogram of the patient, a tissue impedance of the patient, or an intracardiac electrogram of the patient.

Example 18. The system of any one or more of examples 1 to 17, wherein the processing circuitry comprises processing circuitry of the medical device.

Example 19. The system of any one or more of examples 1 to 18, wherein the processing circuitry comprises processing circuitry of a computing device configured to communicate with the medical device.

Example 20. A method comprising: sensing, by one or more sensors of a medical device, one or more sensor signals indicative of one or more physiological parameters of a patient; determining, by processing circuitry of the medical device, based at least in part on the one or more sensor signals, a waveform corresponding to a breathing pattern of the patient, determining, based on the waveform, an envelope signal; determining a sleep disordered breathing index based at least in part on the envelope signal; and determining a heart condition status of the patient based on the sleep disordered breathing index.

Example 21. The method of example 20, further comprising: detecting one or more sleep disordered breathing episodes based on the envelope signal; determining a quantification of one or more sleep disordered breathing episodes; and determining the sleep disordered breathing index based on the quantification.

Example 22. The method of example 21, further comprising activating a sleep study mode of the medical device in response to determining that the quantification of the sleep disordered breathing episodes exceeds a threshold.

Example 23. The method of example 22, wherein the method further comprises: activating, in the sleep study mode, sensing of a first physiological parameter other than respiration; or increasing, in the sleep study mode, a resolution of sensing of a second physiological parameter other than respiration.

Example 24. The method of example 23, further comprising determining the sleep disordered breathing index based on the quantification of the one or more sleep disordered breathing episodes and at least one of the first physiological parameter or the second physiological parameter.

Example 25. The method of example 23, further comprising determining the sleep disordered breathing index by applying a machine learned model to the quantification of the one or more sleep disordered breathing episodes and at least one of the first physiological parameter or the second physiological parameter.

Example 26. The method of example 21, further comprising detecting the one or more sleep disordered breathing episodes based on a difference between the envelope signal and an average of the envelope signal.

Example 27. The method of example 26, wherein the difference comprises a set of difference values for a time period.

Example 28. The method of example 27, wherein the average of the envelope signal comprises a median of the envelope signal over the time period, and wherein the method further comprises calculating each difference value of the set of difference values by subtracting the median value multiplied by a percentage from a corresponding value of a set of values in the envelope signal over the time period.

Example 29. The method of any one or more of examples 26-27, further comprising: determining if a sum of the set of difference values exceeds a threshold; and detecting a sleep disordered breathing episode based on the sum of the set of difference values exceeding a threshold.

Example 30. The method of example 21, further comprising: determining a duration of a time period in which each value of a plurality of values of the envelop signal exceeds a threshold; determining that the time period exceeds a threshold amount of time; and detecting a sleep disordered breathing episode of the one or more sleep disordered breathing episodes based on determining that the time period exceeds the threshold amount of time.

Example 31. The method of example 21, further comprising: determining a phase plot of the envelope signal over the time period; determining if the phase plot shows periodic trends over the time period; and detecting a sleep disordered breathing episode of the one or more sleep disordered breathing episodes based on determining that the phase plot shows periodic trends.

Example 32. The method of example 20, further comprising: collecting an accelerometer signal from an accelerometer of the medical device, wherein the accelerometer signal is indicative of patient movement; determining, based on the accelerometer signal, a time period in which the patient is moving; and determining the sleep disordered breathing index of the patient based on the envelope signal over a portion that does not overlap with the time period.

Example 33. The method of any one or more of examples 20-32, further comprising: determining if the sleep disordered breathing index exceeds a threshold; and saving, in response to the sleep disordered breathing index exceeding a threshold, the waveform over the time period to a memory.

Example 34. The method of any one or more of examples 20-33, further comprising: determining if the sleep disordered breathing index exceeds a threshold; and generating an alert in response to the sleep disordered breathing index exceeding a threshold.

Example 35. The method of any one or more of examples 20 to 34, further comprising determining the heart condition status of the patient based on application of a machine learned model to the sleep disordered breathing index and one or more other physiological parameters of the patient.

Example 36. The method of example 35, where the physiological parameters include one or more of a heart rate of the patient, an activity of the patient, a posture of the patient, a blood pressure of the patient, a body fat percentage of the patient, an electrocardiogram of the patient, a tissue impedance of the patient, or an intracardiac electrogram of the patient.

Example 37. The method of any one or more of examples 20 to 36, wherein the processing circuitry comprises processing circuitry of the medical device.

Example 38. The method of any one or more of examples 20 to 37, wherein the processing circuitry comprises processing circuitry of a computing device configured to communicate with the medical device.

Example 39. A non-transitory computer-readable storage medium having stored thereon instructions that, when executed, cause one or more processors to perform the method of any of examples 20 to 38.

1. A system comprising:
a medical device comprising one or more sensors configured to generate one or signals indicative of one or more physiological parameters of a patient; and
processing circuitry configured to:
determine, based at least in part on the one or more sensor signals, a waveform corresponding to a breathing pattern of the patient;
determine, based on the waveform, an envelope signal;
determine a sleep disordered breathing index based at least in part on the envelope signal; and
determine a heart condition status of the patient based on the sleep disordered breathing index.

2. The system of claim 1, wherein the processing circuitry is configured to:
detect one or more sleep disordered breathing episodes based on the envelope signal;
determine a quantification of the one or more sleep disordered breathing episodes; and
determine the sleep disordered breathing index based on the quantification.

3. The system of claim 2, wherein the processing circuitry is configured to activate a sleep study mode of the medical device in response to determining that the quantification of the one or more sleep disordered breathing episodes exceeds a threshold.

4. The system of claim 3, wherein, in the sleep study mode, the medical device is configured to at least one of:
activate sensing of a first physiological parameter other than respiration; or
increase a resolution of sensing of a second physiological parameter other than respiration.

5. The system of claim 4, wherein the processing circuitry is configured to determine the sleep disordered breathing index based on the quantification of the one or more sleep disordered breathing episodes and at least one of the first physiological parameter or the second physiological parameter.

6. The system of claim 4, wherein the processing circuitry is configured to determine the sleep disordered breathing index by applying a machine learned model to the quantification of the one or more sleep disordered breathing episodes and at least one of the first physiological parameter or the second physiological parameter.

7. The system of claim 2, wherein the processing circuitry is configured to detect the one or more sleep disordered breathing episodes based on a difference between the envelope signal and an average of the envelope signal.

8. The system of claim 7, wherein the difference comprises a set of difference values for a time period.

9. The system of claim 8, wherein the average of the envelope signal comprises a median of the envelope signal over the time period, and wherein the processing circuitry calculates each difference value of the set of difference values by subtracting the median value multiplied by a percentage from a corresponding value of a set of values in the envelope signal over the time period.

10. The system of claim 8, wherein the processing circuitry is configured to:
determine if a sum of the set of difference values exceeds a threshold; and
detect a sleep disordered breathing episode of the one or more sleep disordered breathing episodes based on the sum of the set of difference values exceeding a threshold.

11. The system of claim 2, wherein the processing circuitry is configured to:
determine a duration of a time period in which each value of a plurality of values of the envelop signal exceeds a threshold;
determine that the time period exceeds a threshold amount of time; and
detect a sleep disordered breathing episode of the one or more sleep disordered breathing episodes based on determining that the time period exceeds the threshold amount of time.

12. The system of claim 11, wherein the processing circuitry is configured to:
determine a phase plot of the envelope signal over the time period;
determine if the phase plot shows periodic trends over the time period; and
detect a sleep disordered breathing episode of the one or more sleep disordered breathing episodes based on determining that the phase plot shows periodic trends.

13. The system of claim 1, further comprising an accelerometer, and wherein the processing circuitry is configured to:
collect an accelerometer signal from the accelerometer, wherein the accelerometer signal is indicative of patient movement;
determine, based on the accelerometer signal, a time period in which the patient is moving; and
determine the sleep disordered breathing index of the patient based on the envelope signal over a portion that does not overlap with the time period.

14. The system of claim 1, further comprising a memory in communication with the processing circuitry, wherein the processing circuitry is configured to:
determine if the sleep disordered breathing index exceeds a threshold; and
save, in response to the sleep disordered breathing index exceeding a threshold, the waveform over a time period to the memory.

15. The system of claim 1, further comprising a memory in communication with the processing circuitry, wherein the processing circuitry is configured to:
determine if the sleep disordered breathing index exceeds a threshold; and
generate an alert in response to the sleep disordered breathing index exceeding a threshold.

16. The system of claim 1, wherein the processing circuitry is configured to determine the heart condition status of the patient based on application of a machine learned model to the sleep disordered breathing index and one or more other physiological parameters of the patient.

17. The system of claim 16, where the physiological parameters include one or more of a heart rate of the patient, an activity of the patient, a posture of the patient, a blood pressure of the patient, a body fat percentage of the patient, an electrocardiogram of the patient, a tissue impedance of the patient, or an intracardiac electrogram of the patient.

18. The system of claim 1, wherein the processing circuitry comprises processing circuitry of the medical device.

19. The system of claim 1, wherein the processing circuitry comprises processing circuitry of a computing device configured to communicate with the medical device.

20. The system of claim 1, wherein the medical device comprises an insertable cardiac monitor comprising:
a housing configured for subcutaneous implantation in a patient, the housing having a length between 40 millimeters (mm) and 60 mm between a first end and a second end, a width less than the length, and a depth less than the width;
a first electrode at or proximate to the first end; and
a second electrode at or proximate to the second end,
wherein the insertable cardiac monitor is configured to generate the one or more signals via the first electrode and the second electrode, and
wherein at least a portion of the processing circuitry is disposed within the housing.

21. A method comprising:
sensing, by one or more sensors of a medical device, one or more sensor signals indicative of one or more physiological parameters of a patient;
determining, by processing circuitry of the medical device, based at least in part on the one or more sensor signals, a waveform corresponding to a breathing pattern of the patient;
determining, based on the waveform, an envelope signal;
determining a sleep disordered breathing index based at least in part on the envelope signal; and
determining a heart condition status of the patient based on the sleep disordered breathing index.
